(19) United States
(12) Patent Application Publication
(10) Pub. No.: US 2018/0365825 A1
(43) Pub. Date: Dec. 20, 2018

(54) AUTOMATED PLACENTAL MEASUREMENT

(71) Applicant: Carolyn M. Salafia, Larchmont, NY (US)

(72) Inventor: Carolyn M. Salafia, Larchmont, NY (US)

(21) Appl. No.: 15/949,020

(22) Filed: Apr. 9, 2018

Related U.S. Application Data
(63) Continuation-in-part of application No. 12/573,616, filed on Oct. 5, 2009, now abandoned.

(51) Int. Cl. G06T 7/00; G06K 9/00; G06F 19/12; G06T 19/20; G16H 30/40

(52) U.S. Cl. CPC G06T 7/0012; G06K 9/0014; G06F 19/12; G06T 19/20; G06T 2207/10056; G06T 2207/30024; G06T 2207/30044; G06T 2207/30104; G16H 30/40

(57) ABSTRACT

The present invention teaches a method of predicting the potential for manifestation of various medical conditions by analyzing human placenta. The method contemplates and includes determining the need for early monitoring, intervention or potential treatment for medical conditions likely to manifest as a child grows older and investigating the potential for various medical conditions. The method includes selecting and identifying a sample of the placenta to analyze by computer applied mathematical algorithms and preparing the placental sample to be analyzed by various procedures. Then the placental sample is captured by obtaining a three-dimensional digital image of the chorionic surface of the placental sample by use of a selected capturing device. The digital image is corrected for errors inherent in digital image acquisition and the resultant image data is loaded into a computer for analysis. The computer performs an analysis on the corrected digital image data using one or more algorithms to determine the vascular structure of the placenta, and the resultant data is interpreted and analyzed to determine the potential for manifestation of various medical conditions.

### REFERENCE TO RELATED APPLICATIONS

This application is a continuation-in-part of U.S. application Ser. No. 12/573,616, filed Oct. 5, 2009.

### STATEMENT REGARDING FEDERALLY SPONSORED RESEARCH OR DEVELOPMENT

Not Applicable

### INCORPORATION BY REFERENCE OF MATERIAL SUBMITTED ON A COMPACT DISC

Not Applicable

### FIELD OF THE INVENTION

The present invention generally relates automated imaging measures of the intrauterine environment through measures of placental imaging and histology.

The placenta, the key organ upon which the fetus is entirely dependent for all oxygen and nutrition, grows in a branching fashion analogous to the growth of a tree and its branches. The major villous types, their principal time periods of development during gestation, and their specific physiology have been well delineated in the research setting. But routine pathology slide review has poor reliability in distinguishing the major patterns of placental branching morphogenesis. As the evidence that lifelong health risks appear to be correlated with birthweight, the importance of placental growth and development as the principal non-genetic contributor to fetal growth has grown.

The placenta is the only fetal organ that can be dissected in a living child to yield information related to cell proliferation (a marker of tissue health), branching (reflecting gene transcription events) and cell death.

Placental vascular growth, essential to healthy fetal life, is too complex to be reliably estimated even by specialists. Indeed, pathologists often make unreliable diagnoses of histology features that are recognized to be associated with long term health risks.

A reliable and automated assessment tool performed on routine stained placental slides may help understand how intrauterine stressors modulate placental (and by extension fetal) well-being.

Thus, there exists the need for an automated, reliable, and inexpensive method of measurement of placental vascular growth through placental imaging and histology.

### BRIEF SUMMARY OF THE INVENTION

Disclosed herein is a new approach towards automated measures of the intrauterine environment through placental imaging and histology.

Other objects and features will be in part apparent and in part pointed out hereinafter.

### BRIEF DESCRIPTION OF THE SEVERAL VIEWS OF THE DRAWING

FIG. 1 is a photograph of a histological tissue cross section of the neutrophils.

FIG. 2 is a photograph of a histological tissue cross section of a pair of before and after images of placental tissue processed by a spatial fuzzy c-means algorithm (SFCM algorithm).

FIG. 3 is a photograph of a histological tissue cross section of a pair of before and after images of placental tissue processed by a spatial fuzzy c-means algorithm (SFCM algorithm). In this application of the algorithm the parts of the image that show views of the connective tissue have been extracted.

FIG. 4 is a group of six digital histological, photographs of placentas that have been analyzed and the umbilical cord centrality measured.

FIG. 5 A is a histological, digital image of a placenta.

FIG. 5 B is a histological, digital image of placentas cut into seven sections.

FIG. 5 C is a histological, digital image of a placenta used for comparison with a reconstructed, 3D image of the placenta.

FIG. 5 D is a reconstruction of the three dimensional image by use of mathematical algorithms.

FIG. 6 is a series of histological, photomicrographs depicting selected photomicrographs from cases with extreme low (negative) and high (positive) factor scores.

FIG. 7 is a digitized, histological photograph of the placental chorionic surface marked for the umbilical cord insertion, the disk perimeter (outer marking), and terminal points of chorionic vascular plate branches (inner marking).

FIG. 8 is a histological, digital image showing the chorionic surface vessels traced by hand.

FIG. 9 A is a histological, digital images showing the chorionic surface of a placenta.

FIG. 9 B is a histological, digital image showing the chorionic surface of the placenta shown in FIG. 9 A traced by hand.

FIG. 9 C is a histological, digital image showing the chorionic surface of the placenta shown in FIG. 9 A with the vessels extracted using a neural net algorithm.

FIG. 10 A-E are a series of schemas depicting determination of chorionic plate area and centroid, chorionic vascular area and centroid, and discordance or concordance of centroids.

FIG. 11 A is a histogram depicting the distribution of baby weight.

FIG. 11 B is a histogram depicting the distribution of placental weight.

FIG. 11 C is a histogram depicting the distribution of inter-centroid distance normalized to chorionic plate area.

FIG. 12 is a histology slide which demonstrates the capacity for the 3-D reconstruction of the placental finer villous tree.

FIG. 13 A is a histological photograph illustrating the addition of barium infusion into the placental sample.

FIG. 13 B is a histological photograph illustrating the addition of barium infusion into the placental sample with shearlet-smoothed.

FIG. 13 C is a histological photograph illustrating the addition of barium infusion into the placental sample with shearlet-smoothed and Laplacian Eigenmaps enhanced.

FIG. 13 D is a histological photograph illustrating the addition of barium infusion into the placental sample with shearlet-smoothed, Laplacian Eigenmaps enhanced, after final thresholding.

### BRIEF DESCRIPTION OF THE DRAWINGS

Those of skill in the art will understand that the drawings, described below, are for illustrative purposes only. The drawings are not intended to limit the scope of the present teachings in any way.

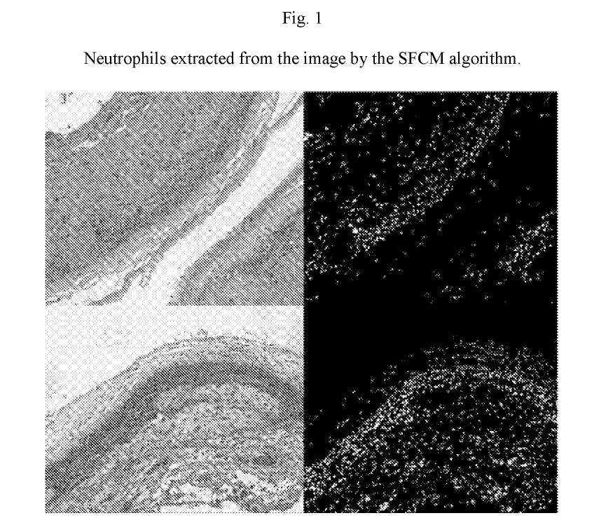

FIG. 1 is a pair of before and after images of placental tissue processed by a spatial fuzzy c-means algorithm (SFCM algorithm). In this application of the algorithm the parts of the image that show the neutrophils have been extracted.

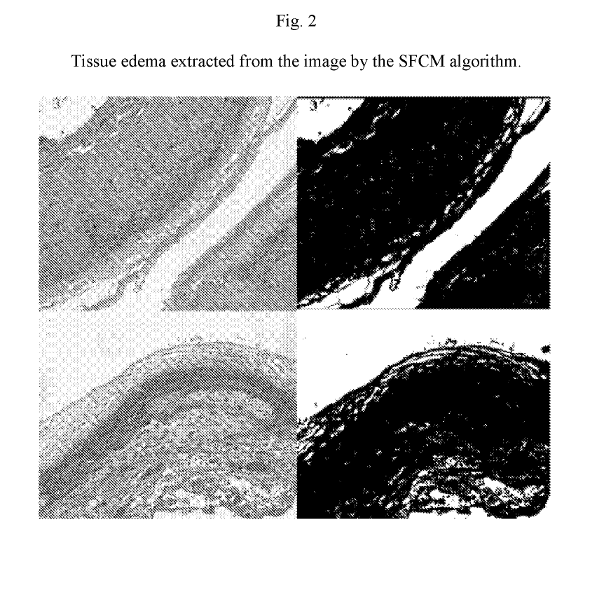

FIG. 2 is a pair of before and after images of placental tissue processed by a spatial fuzzy c-means algorithm (SFCM algorithm). In this application of the algorithm the parts of the image that show an edema of the connective tissue have been extracted.

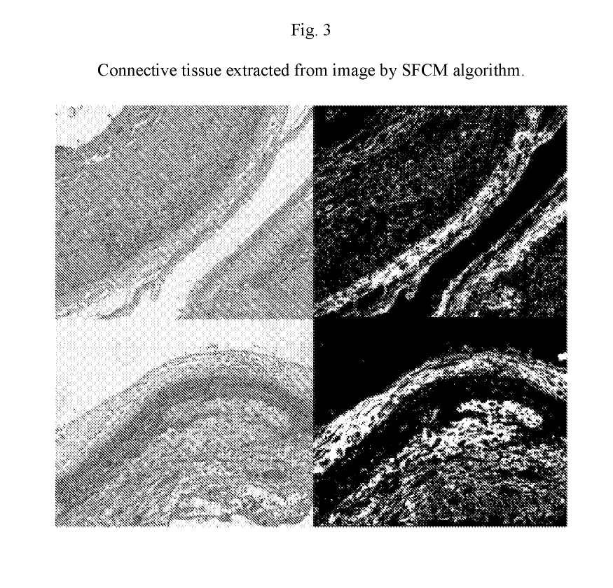

FIG. 3 is a pair of before and after images of placental tissue processed by a spatial fuzzy c-means algorithm (SFCM algorithm). In this application of the algorithm the parts of the image that show views of the connective tissue have been extracted.

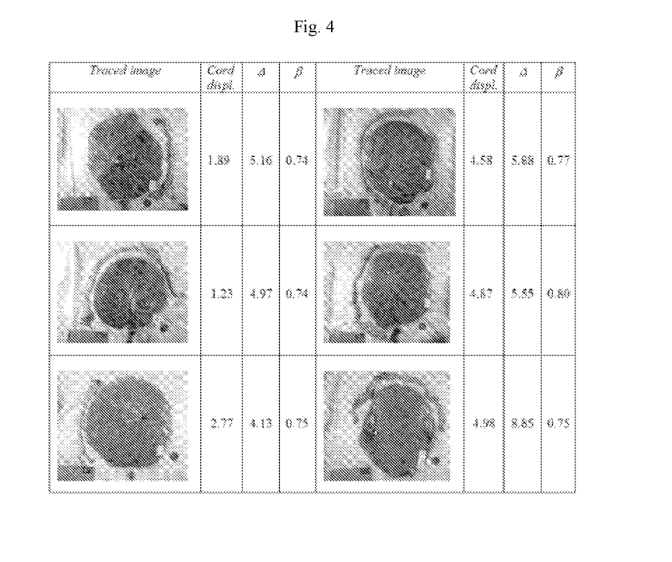

FIG. 4 is a group of six digital images of placentas that have been have been analyzed and the umbilical cord centrality measured.

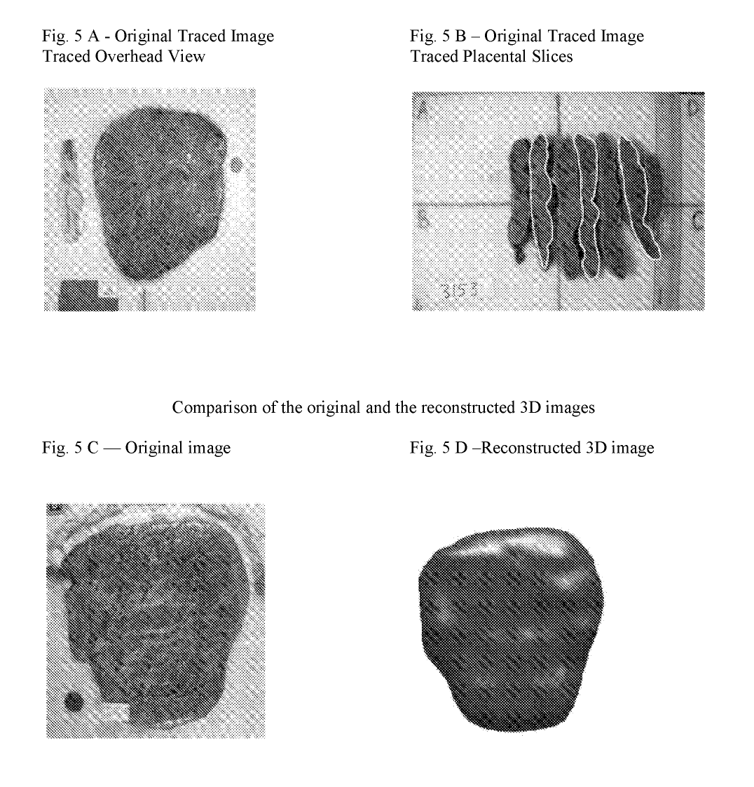

FIG. 5 A, FIG. 5 B, FIG. 5 C, and FIG. 5 D are a group of 4 digital images of a placenta, placentas cut into seven sections, and a reconstruction of the three dimensional image by use of mathematical algorithms.

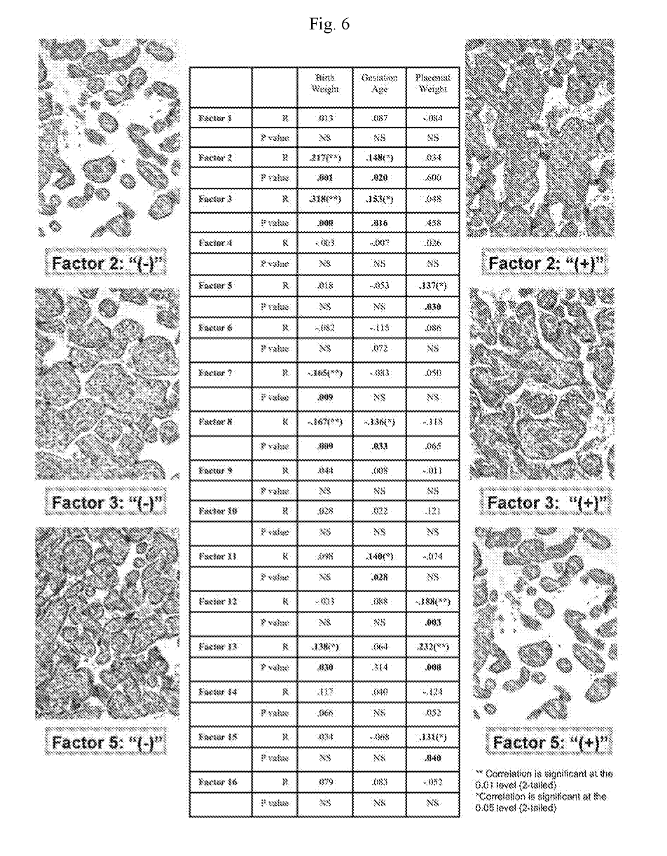

FIG. 6 is a series of photomicrographs depicting selected photomicrographs from cases with extreme low (negative) and high (positive) factor scores.

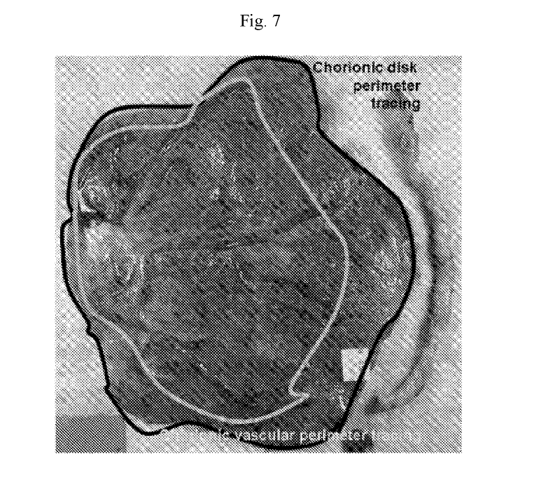

FIG. 7 is a digitized photograph of the placental chorionic surface marked for the umbilical cord insertion, the disk perimeter (outer marking), and terminal points of chorionic vascular plate branches (inner marking). The ratio of these areas is associated with decreased observed/expected birth weight.

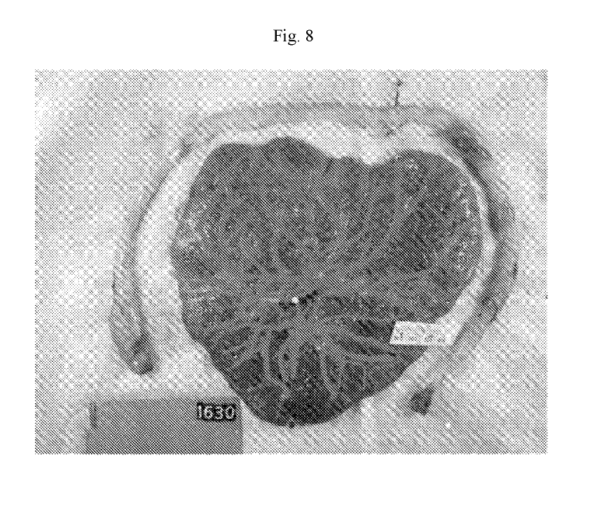

FIG. 8 is a digital image showing the chorionic surface vessels traced by hand.

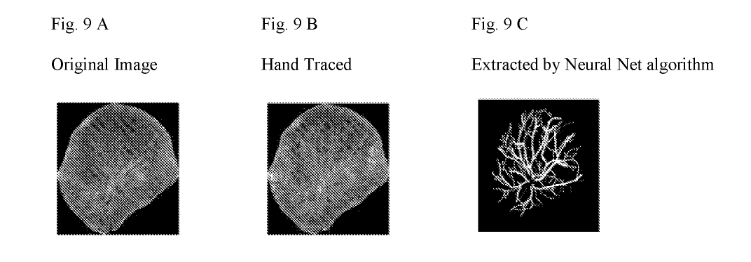

FIG. 9 A, FIG. 9 B, and FIG. 9 C are a group of three digital images showing the chorionic surface, the same surface with the vessels traced by hand, and the same surface with the vessels extracted using a neural net algorithm.

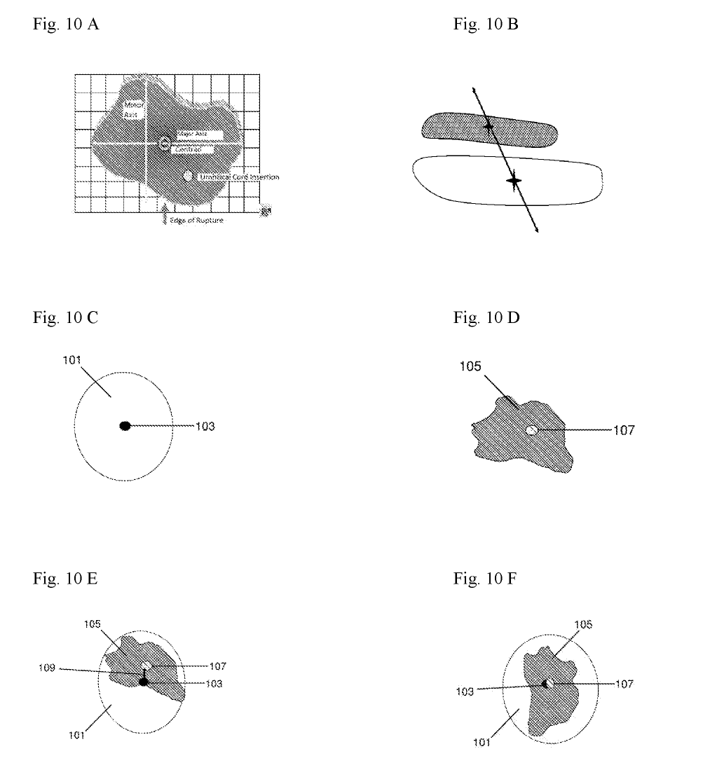

FIG. 10 A, FIG. 10 B, FIG. 10 C, FIG. 10 D, FIG. 10 E, and FIG. 10 F are a series of schemas depicting determination of chorionic plate area and centroid, chorionic vascular area and centroid, and discordance or concordance of centroids. FIG. 10 A shows a schematic of a placenta as it is fixed for analysis. The macro used calculates areas, and the centroid, the weighted center of the area. The chorionic plate and the chorionic vascular area are essentially treated as a pair of shapes that should “fit.” “Fit” is reflected in the distance between centroids as is shown schematically in FIG. 10 A through FIG. 10 F. FIG. 10 C shows the chorionic plate area 101 and chorionic plate area centroid 103. FIG. 10 D shows the chorionic vascular area 105 and chorionic vascular area centroid 107. The centroids of the two shapes may be discordant or concordant. The inter-centroid distance is limited by the chorionic plate size. The inter-centroid distance is normalized for chorionic dimensions. FIG. 10 E is a schematic showing discordant centroids with a long inter-centroid distance 109. FIG. 10 F is a schematic showing concondant centroids.

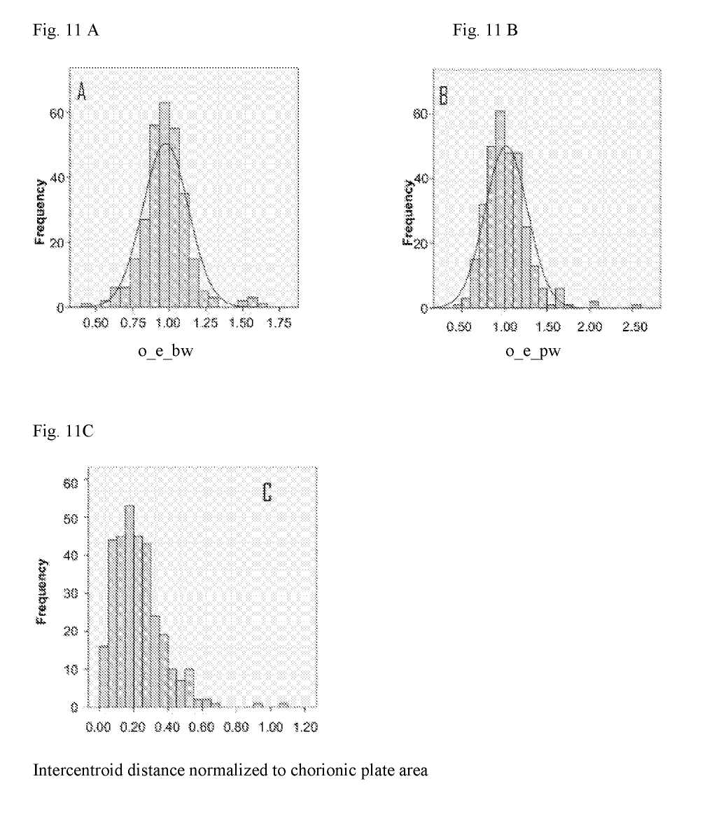

FIG. 11 A is a histogram depicting the distribution of baby weight. FIG. 11 B is a histogram depicting placental weight. FIG. 11 C is a histogram depicting inter-centroid distance normalized to chorionic plate area.

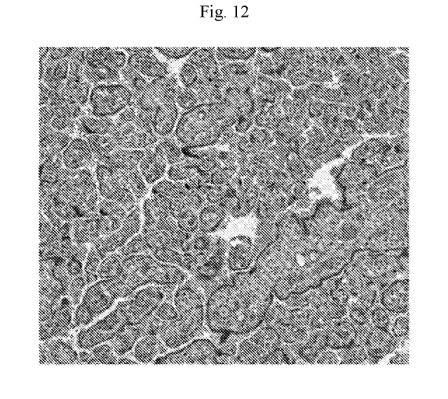

FIG. 12 is a histology slide which demonstrates the capacity for the 3-D reconstruction of the placental finer villous tree.

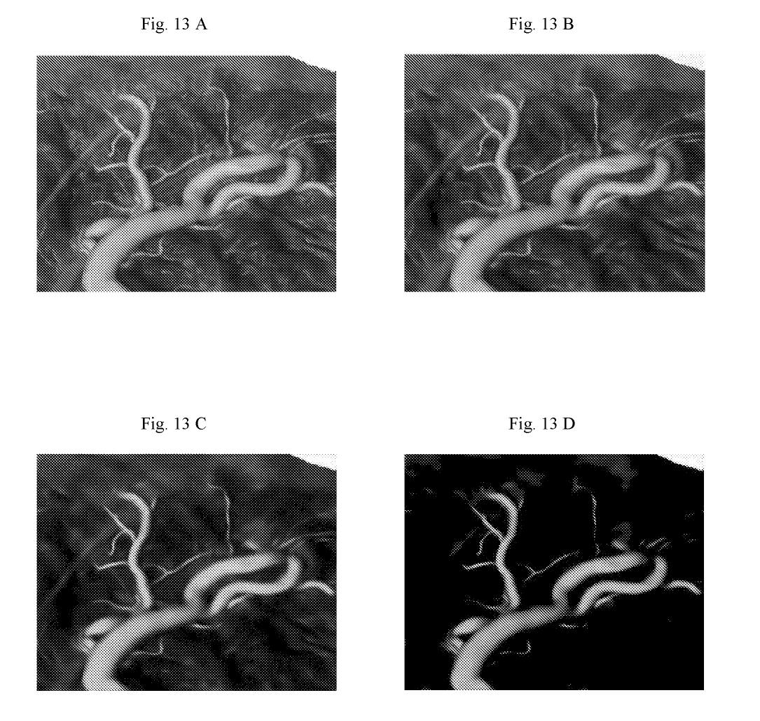

FIG. 13 A through FIG. 13 D are a series of images illustrating the addition of barium infusion into the placental sample. FIG. 13 A illustrates shearlets captured directional and curved structures in the image, and FIG. 13 B shows the same image after smoothing structures away from the vessels. The Laplacian Eigenmaps differentially emphasized vessel structures at different scales, shown in FIG. 13 C. Intensity thresholding further enhances the image contrast as shown in FIG. 13 D. As is apparent from the image, noticeably, superior smoothing is observed on fixed images in non-vessel-like regions due to the lack of speckles induced by glare and blood, as shown in FIG. 13 D.

### DETAILED DESCRIPTION OF THE INVENTION

The methods described herein teach a process for extracting medically significant information from digital images of placentas and placental tissues by processing the image through a mathematical algorithm. The medically significant information extracted from the image may, for example, count neutrophils that are responses to bacterial infection. High neutrophil counts indicate a bacterial infection was present in the fetal environment before birth. Such bacterial infections are one of the most significant predictors of risk for Cerebral Palsy (CP) in term infants. Cerebral Palsy is not diagnosed until several years after birth; CP cannot be identified by examination of the mother or the newborn, but identification of the risk of CP by the methods taught herein can enable a physician to prescribe a plan of monitoring and early intervention if signs of the disease begin to manifest themselves. An example of the extraction of neutrophil information is discussed further in Example 1.

Another example of medically significant information that can be extracted from digital images is a measure of the integrity of the connective tissue. As a result of bacterial or viral infections, these connective tissues may be damaged by digestive enzymes released by the neutrophils recruited to attack the invader. These same enzymes can damage the connective tissues of the fetus and lead to brain and lung damage in the child. As with CP, this damage may not be observable in the newborn, but the information produced by these methods for analysis of the placenta may allow a physician to prescribe early monitoring, intervention, or treatment for the infant and child. Furthermore, medically significant information can extracted from digital images of placental histological features so as to provide analysis of congenital viral infection (well recognized as a precursor to fetal anomalies as well as poor long-term neurological development), and maternal/uteroplacental and fetal-placental vascular pathologies (both of which are associated with fetal hypoxia and risk for poor long-term neurological development).

Another example of medically significant information is the measurement of placental shape that measures the underlying vascular fractal and is an indirect measure of healthy placental growth throughout pregnancy and indicates times during pregnancy when stressors alter placental (and by extension, fetal) health. Placental shapes can be measured by image segmentation or pixel counting and Fourier analysis.

Another example of medically significant information is the quantitation of chorionic branching structure. The number of chorionic blood vessels, the number of branch points, inter-branching intervals, and the total vascular length are measured to quantify aspects of vascular growth and gene events relevant to fetoplacental branching and growth early and throughout gestation. Segmentation and branching metrics including Laplacian and other graph and network metrics can be used to analyze the 2-dimensional image to quantitate and time the severity and numbers of events contributing to deformation of placental vascular branching growth.

Another example of medically significant information is the Fourier analysis of placental shape that indicates the time and severity of deformed placental vascular growth, and quantitates the effect of altered placental shape on placental function through effects on placental scaling.

Another example of medically significant information is the assessment of villous maturation and potential exposure to hypoxia, congenital viral infection, fetal vascular pathology, and abnormal maternal uteroplacental perfusion. Altered villous size, vascularity, extent and integrity of connective tissue, number, hue and intensity of syncytial and stromal nuclei can be used to measure appropriate placental maturation and also serve as indicators of villous diseases that affect placental function and/or fetal health.

Another example of medically significant information is the 3-dimensional reconstruction of the gross placental shape and its mathematical solution, the inverse of which represents the maternal intrauterine environment. Fourier analysis of placental shape indicates the time and severity of deformed placental vascular growth, and quantitates the effect of altered placental shape on placental function through effects on placental scaling.

Another example of medically significant information is the 3-dimensional reconstruction of the villous stem vascular tree. The 3-dimensional reconstruction may be obtained by mathematical recombination of two or more serial sections. Segmentation or branching algorithms can be used to “prune” or remove the finer villous elements leaving the larger branches for analysis. The number of fetal stem blood vessels, the number of branch points, inter-branching intervals, and the total vascular length are measured to quantify aspects of vascular growth and gene events relevant to fetoplacental branching and growth early and throughout gestation. Segmentation and branching metrics including Laplacian and other graph and network metrics can be used to analyze the 3-dimensional image to quantitate and time the severity and numbers of events contributing to deformation of placental vascular branching growth.

Another medically useful technique is the analysis of individually segmented villi for their maternal/uteroplacental and fetoplacental functions using standard diffusion equations.

Another example of medically significant information is the measurement of the timing of the occurrence of events or stressors that affect the growth and development of the placenta and the fetus. The influence of these events or stressors can manifest themselves in the development and branching of the placental vascular system. These events or stressors cause the vascular system to develop in ways that make it deviate from its nominal fractal scale and different types of deviations from the nominally round shape can indicate an earlier event. Thus, measurements of the placental vasculature or the placental shape using algorithms such as segmentation or branching metrics including Laplacian and other graph and network metrics can reveal information about when during the development of the placenta changes occurred that altered or influenced its development. Also, determination of which blood vessels have been affected can lead to an assessment of timing. For example, the chorionic vessels are developed early in pregnancy, and so events that change their development therefore must have occurred early in pregnancy.

The timing of events that change the development of the placental vasculature are correlated with fetal characteristics that are, in turn, strongly associated with childhood health risks. For example, it is commonly understood by those of typical skill in the art that birth weight is a primary indicator of childhood health risk. As birth weight deviates from the optimum range, the risk of childhood health issues increases. Similarly, it is understood that placental weight is strongly correlated with birth weight, and deviations from that correlation are also associated with childhood health risks. The inventors have discovered that placental vascular branching affects placental efficiency and affect birth weight independently of the placental weight.

Yet another example of medically significant information is the assessment of timing of placental infection. The duration of an infection can be determined by the effects of bacterial and bodily-produced chemicals on many different cell types in the placenta, cord and membranes. One example of effect on infection on these tissues is the infiltration of neutrophils that combat pathogens into the placental tissues. Other cells affected by infection and its related physiology include epithelia, connective tissue and fibroblasts, monocyte/macrophages, and vascular endothelia. For example, segmentation algorithms disclosed herein are useful in extracting the images of neutrophils from the digital image of the placental histology slides. As a further example, mathematical analysis, using algorithms that compute the mean distance of each particle to the placental surface, provide an assessment of time of infection.

The first step of these methods is the selection of the placental sample to be analyzed. Every baby is born with a placenta and the sample may be of the entire placenta (i.e., a digital image) or taken from the placenta, the umbilical cord, or the membranes. The sample may be the entire placenta, the gross placental shape, portions of the placenta, umbilical cord, or membranes, or may be a slice of tissue from any of these fixed to a histology slide. The samples may be taken soon after birth, or the placental tissues may be preserved in formalin and the digital images may be taken at a later date, even years later. Measurements taken at birth can be used to predict risk to future pregnancies born to that mother, as well as risks to the particular child.

A digital image of the placental sample may be obtained using a film or digital camera, using a microscope with a camera attachment, or using a slide digitizer. Film images may be digitized if they are of sufficient resolution. For obtaining a digital image of the entire placenta, the preferred method is to use a digital camera. For obtaining a digital image of histology slides, the preferred method is to use a slide digitizer such as an Aperio T3, manufactured by Aperio Technologies Corp. in Vista, Calif. Other slide digitizers may be used such as those manufactured by Nikon, Zeiss, or Leica. The digital images of histology slides should have sufficient resolution to allow extraction of image features up to a magnification of 20-40×.

The digital images are analyzed by processing them by a mathematical algorithm. Several types of algorithms may be employed alone or in combination to extract the features of interest from the image. Among the algorithms that can be used are spatial fuzzy c-means algorithms, segmentation algorithms, boundary finding algorithms, counting algorithms, length measuring algorithms, branching algorithms, angle measuring algorithms, and color discriminating algorithms. Other types of algorithms useful for image analysis or segmentation are clustering (K-means) algorithms, mean shift algorithms, histogram-based algorithms, edge detection algorithms, region growing algorithms, level setting algorithms; graph partitioning algorithms, watershed transformation algorithms, model based segmentation algorithms, multiscale segmentation algorithms, semi-automatics segmentation algorithms, and neural network algorithms. For example, a branching algorithm may be used to extract the extent of branching of the major placental blood vessels from the digital image of the chorionic surface of the entire placenta. A color discriminating algorithm may be used to extract the neutrophils from a digital image of a histology slide and then a counting algorithm used to count the number of neutrophils present.

These mathematical algorithms analyze the image by the application of mathematical rules. For example, one particularly useful algorithm is the spatial fuzzy c-means (SFCM) algorithm. The unsupervised cluster algorithm, called SFCM (Spatial Fuzzy c-Means), is based on a fuzzy clustering c-means method that searches the best fuzzy partition of the universe assuming that the evaluation of each object with respect to some features is unknown, but knowing that it belongs to circular regions of R 2 space. The spatial function is the summation of the membership function in the neighborhood of each pixel under consideration. The advantages of the SFCM are the following: (1) it yields regions more homogeneous than those of other methods, (2) it reduces the spurious blobs, (3) it removes noisy spots, and (4) it is less sensitive to noise than other techniques. This technique is a powerful method for noisy image segmentation and works for both single and multiple-feature data with spatial information.

The features of interest include neutrophils, connective tissues, portions of edema, cell nuclei, major blood vessels, branched villi, large villi, long villi, small villi, nutrition exchange vessels, and capillaries, markers of fetal hypoxia such as syncytial knots and syncytial basophilia, villous fibrosis/scarring, chronic villitis and chronic intervillositis, infarcts, abruption, perivillous fibrin deposition and cytotrophoblast proliferation, abnormalities of clotting and inflammation in the basal plate and maternal uteroplacental vessels, cell death of epithelia, stroma, endothelia, proliferation of macrophages and fibroblasts in connective tissue and stroma, abnormalities of clotting and inflammation in the fetal-placental blood vessels.

After extracting the feature of interest from the digital image measurements of those features may be made and statistics of those parameters may be calculated. In one example noted above the neutrophils can be extracted and then counted. Similarly, syncytial knots may also be extracted and counted. The major blood vessels may be extracted and their lengths and areas measured with statistics such as minimum, maximum, and mean computed.

Obtaining the digital image, analyzing the image, extracting the features of interest, applying the algorithm or algorithms, and computing relevant statistics may be automated by computer scripts or macros. The physician or pathologist will insert a slide in a slide digitizer and via an interface select features of interest or regions of interest on the image. If it is necessary, the physician may use one or more additives to facilitate the analysis on the sample. For example, barium may be added to improve the contrast between the vascular structure of the placenta and the surrounding tissues. Once the sample is ready to be analyzed, the pertinent information is entered by the physician and computer scripts will perform the requested analysis, reporting the relevant measurements or statistics in an automated operation. It is contemplated within the scope of this invention that these scripts may allow a slide to be inserted into the slide digitizer and the computer will look for any evidence of abnormality or disease in a completely automated operation without prior physician input.

It will be readily appreciated by those skilled in the art that the process is contemplated to be automated, however, depending on the characteristics of the individual sample, more input from the physician may be necessary, particularly in the steps preparing the sample to be analyzed.

Statistics derived from the digital image are correlated with known health risks and outcomes. High numbers of neutrophils are known to be related to risk of Cerebral Palsy. Vascular edema is related to brain damage. Lack of integrity of connective tissue is related brain, lung, and heart damage. Additional published studies relate the health and development of the fetus, as reflected by changes in birth weight that are independent of parental or extrauterine factors, to the long term health—or health risks—of children and adults. The placenta, as the fetus' sole source of oxygen and nutrients, is the principal determinant of fetal growth independent of factors such as parental size and reflects the adequacy of the maternal environment.

Reliable measures of placental tissues as taught by the methods described herein enable physicians to more accurately assess future health risks, risks to future pregnancies of that mother, and to prescribe monitoring, intervention, and treatment at an earlier time and to greater effect of her current child. Thus is provided an approach for an automated and method of placental diagnosis that includes a completely novel measurement of placental vascular branching structure and more comprehensive and reliable histopathology diagnoses that can be performed on a routine hematoxylin and eosin stained slide obtained from, for example, the placenta at birth. This measure can improve diagnosis of fetal growth restriction, identify critical periods of abnormal placental growth that might mark risks for later health risks, and reliably diagnose placental histopathology features that have been associated with increased long term neurodevelopment risks but which remain unreliably diagnosed by routine pathology. The measure is comprehensive, including both measures of the whole placenta and visible features of the chorionic surface vasculature with measures of the fine (microscopic) placental structure. Further, the measurement is automated, incorporating into its algorithms the full field of knowledge of placental structure, pathology, and functional correlates. The reliability of the method and the ease of preparation of a routine stained slide, makes its application practical on a wide population basis. As such, the diagnoses generated by these measurement tools would be accessible to all newborns. Such tools could impact public health burdens as obesity and diabetes, cardiovascular disease, certain cancers, and psychological disorders, disorders that have their genesis, at least in part, in fetal life.

Image Analysis: Gross Placental Features.

Image segmentation methods are described herein to be applied to the gross features of the placenta and to histology slides taken from placental tissues. The prior art method for measuring the whole placenta involves describing whether the placenta is round/oval or more irregular, noting whether more than one placental lobe is present, and taking a single measurement of larger and smaller diameters, and a single measure of the placental disc thickness. This method may be used in capturing the shape of regular, round/oval placentas, but is unreliable in regards to the irregular placental shapes that are commonly considered to reflect the effects of the most problematic maternal/uteroplacental environments and the formations of normal placental growth patterns. We have demonstrated, using the publicly available data collected as part of the Collaborative Perinatal Project, that abnormal placental shape has a persistent negative effect on birthweight after adjustment for placental weight and other placental dimensions. Thus, given two placentas, each weighing 500 g, the placenta with the irregular shape will yield a statistically significantly smaller baby than a round/oval placenta. This means that abnormal placental shape is not compensated for by further placental growth. Furthermore, abnormal placental growth affects the three-quarter scaling of placental growth to fetal growth, indicating that these abnormal shapes reflect abnormal placental vascular fractal networks. While most normal placentas (placentas delivered with infants who are well grown at term and not admitted to the neonatal intensive care unit) will have a uniform thickness, many placentas have variable thickness which is well recognized to reflect variable arborization of the placental villous trees. It is generally held that such variability in villous arborization reflects maternal uteroplacental pathology. However, current surgical diagnostic methods do not capture variability in disc thickness, and current research methods cannot allow such variability to be analyzed.

We described that more precise measurement of placental perimeters increases the total amount of birthweight variants attributable to placental factors. However, this measurement method required a trained technician applying costly software, and could not be used on a population basis. Our current methods involve the simple tracing of the placental perimeter, noting appropriate landmarks (such as umbilical cord insertion and the edge of the placenta closest to the free edge of the ruptured membranes) with a drawing tool in Adobe Photoshop. Use-specific algorithms written in MatLab code extract a series of quantities that reflect the area, eccentricity, and regularity of the shape. We have applied the same method to marking the vascular parameter of the chorionic disc, to furthest-most extensions of the chorionic surface vessels on the plate. A similar use-specific algorithm calculates a series of quantities, and the two sets of quantities are used to calculate the eccentricities, among other features, of the two shapes.

Umbilical Cord Centrality

The current art for measuring the insertion, or connection point, of the umbilical cord into the chorionic surface of the placenta, is to measure the distance from the edge of the umbilical cord insertion to the nearest edge of the placenta. The selection of the nearest edge point is done by eye, and the measurement is taken to the nearest centimeter.

The inventor has discovered that the location of the insertion of the umbilical cord is an important indicator of abnormal growth and development of the placenta, and, in turn, the potential for abnormal growth and development of the fetus. The inventor believes, without wishing to be bound to a particular theory, that this surprising discovery may be due to the development of the fractal growth of the system of blood vessels in the placenta. An umbilical cord insertion that deviates from the geometric center of the placenta (regardless of the shape of the placenta) reflects the result of abnormal force or forces acting on the fractal growth of the placenta, deforming the fractal. The deformation of the fractal growth leads to abnormal growth and development of the placenta which causes it to be less than optimal in structure and less than optimal in its function of delivering oxygen and nutrients to the fetus.

The current art for measuring the insertion, or connection point, of the umbilical cord into the chorionic surface of the placenta, is to measure the distance from the edge of the umbilical cord insertion to the nearest edge of the placenta. The selection of the nearest edge point is done by eye, and the measurement is taken to the nearest centimeter. This measurement is inadequate in many ways. Placentas may be circular, elliptical, multi-lobed or irregular in overall shape. A measurement to the nearest edge does not reveal where on the surface the umbilical insertion actually is, nor does it reveal the location of the insertion point with regard to the placenta's geometric center.

The inventor has discovered that these difficulties can be overcome by the automated analysis of digital images of the placental chorionic surface using one of a group of mathematical algorithms. One example of a mathematical algorithm is the use of Fourier analysis of the radial distances from the umbilical cord insertion point to a point on the placental perimeter as the computer sweeps around the perimeter, analyses the deviation of the umbilical cord insertion point from the calculated geometric center. Another example of a mathematical algorithm is the computer measurement of the radial distances from the umbilical cord insertion point to a point on the placental perimeter as the computer sweeps around the perimeter. The radial distances are plotted as a function of the sweep angle theta, and the first and second derivates of the function are computed.

A measure of the centrality of the cord using a Fourier analysis is obtained as follows. First, the umbilical insertion point is placed at the origin. Perimeter markers are connected by straight line segments to obtain an approximate perimeter P of the chorionic plate. A sector of opening of 6° with vertex at the origin is rotated in 6° increments. For each turn of the sector, the points in P inside of it are averaged to yield a radial marker. In this way, we obtain 60 radii emanating from the origin spaced at 6° intervals. They are connected to obtain the angular radius r(θ), which is a function of the angle θ from the umbilical insertion point. The function r(θ) can be analyzed using the standard techniques of Fourier series. In particular, we computed the first Fourier coefficient of r(θ).

The first Fourier coefficient, |C|, can be used as a measure of the centrality of the umbilical cord. It measures the “average oscillation” of the placental radius in one full turn around the umbilical insertion point.

Cord centrality significantly impacts placental efficiency: non-central cord insertion for the same placental weight results in a smaller baby. We note first, that placentas with larger value of the cord displacement tend to be larger in size. The value of the cord displacement found from analysis of photographs taken from a birth cohort collected by the University of North Carolina was correlated with the mean placental radius (correlation 0.046) and with the placental weight (correlation 0.164). To determine if the placentas with a large cord displacement were as metabolically efficient as the normal ones, we have calculated the correlation of cord displacement with the scaling exponent:

β=log(Placental Weight)/log(Birth Weight).

It is large (0.158) and very significant (0.000). When we use the size of the first Fourier coefficient |C| as the measure of the cord displacement, the correlation with is even larger (correlation 0.2, significance 0.000). Thus, the placentas with a large umbilical cord displacement, measured either as a distance from the geometric center, or as |C|, are less metabolically efficient (FIG. 4). Even though these placentas grow larger than normal, the added placental weight does not translate into the corresponding gain for the birth weight. Placentas with a non-centrally inserted cord tend to produce smaller babies than normal placentas of the same weight.

Thus, non-central insertion of the umbilical cord is a source of deformation of the macroscopic placental architecture. This is somewhat unexpected, as the shape of a placenta with a non-central insertion can still be round, as confirmed both by our statistical analysis, and by the dynamical models of placental growth. Even if typically a placenta with a non-central insertion is of a normal round shape, its surface vascular distribution is sparse and, as reflected by a larger β is less metabolically effective. This results in a smaller birth weight for the same placental weight.

The altered structure of the surface vasculature can be seen from measurements of the coverage of the placental surface with the large branches of the vascular tree. The placentas with a non-centrally inserted cord suffer from a sparser vascular coverage, so that a point on the surface is typically further away from a large blood vessel than in a normal placenta. But the easiest-to-grasp indicator of the deformation of the placental vascular architecture as a whole (both macroscopic and microscopic finer structure of placental stem and terminal villi) is the metabolic scaling exponent β calculated as the ratio of the logarithms of the placental weight and the baby birth weight. The quantity 1/β should be seen as a biologically relevant version of the fractal dimension of placental vasculature, so the larger value of β implies a poorer placental functional efficiency and an altered placental vascular fractal. We observe that the value of β is strongly and significantly correlated with non-centrality of the cord insertion. Placentas with a non-centrally inserted umbilical cord are typically larger both in diameter and by weight. Without wishing to be bound to a particular theory, we speculate that the larger size is a compensatory mechanism for a reduced efficiency per unit of placental weight.

3D Reconstruction of the Placental Shape.

The placental disc is fixed in formalin and subsequently sliced in eighths, and the seven unique surfaces are digitally photographed. These slices are used to add n “height function” to the surface information.

Currently, the most common measurement of the placenta is its weight, which has been shown to correlate with infant and childhood health risks. Crude measurements of placental surface dimension (usually a largest and smallest diameter) are also routinely made. Measurements of the volume are not routine, but can be made by water displacement. None of these, however, reveal the 3-dimensional shape of the placenta, which is an important indicator of the development of the fine vascular structure. Placentas have been sliced through their depth into four or more pieces to obtain an estimate of the 3-dimensional structure, but a need exists to re-assemble the digital images of the slices to reconstruct the entire 3-D shape.

The inventor has discovered a method to characterize the shape of a placenta using geometric descriptors derived from a reconstructed surface. Given the traced coordinates of the overhead image and individual slices in two dimensions, we implemented algorithms that translate these data into three dimensions. More than one type of algorithm that reconstructs a surface based on three dimensional data can be used. In one embodiment, we applied one of two groups of surface reconstruction methods: implicit and explicit methods. The explicit method hinges on the specific ordering of the traced data and is notably fast. The first implicit method, based on the Level Set Method can be applied to any unorganized set of points. This 3D level set method shrinks an initial guess to a smooth surface on the sample points. In another embodiment we partially implement the method of contour metamorphosis which is based on a 2D level set method. Geometric descriptors, such as surface areas, volumes and medial axes, are computed based on these reconstructed models.

The data come in two parts: coordinates on cross-sections of the placenta, and coordinates on the outline of the placenta as viewed from above. These coordinates were collected by hand, by tracing a digital image of the placenta and its cross-sections on a Kurta drawing tablet. In addition, the way in which the cross-sections were cut is known. There are three different sets of data, each with a different cutting method. Any placenta with greater than seven slices was cut at one centimeter intervals. All placentas with seven slices were cut in half, then the pieces were cut in half, and once more those pieces were cut in half. The placentas with only five slices were cut at one centimeter intervals as well, but only five slices in the middle of the placenta were collected.

Dense Data.

We also applied these methods to data in the form of traced photos of slices and overhead images of a select group of placentas (FIGS. 5A and 5B). We refer to them as dense data. The overhead image consists of the original overhead placental image with a green line traced by hand around the contour of the placenta. There is also a yellow dot indicating the cord insertion site. The slices image consists of the original image with alternating red and yellow traces around the contour of the slices. Each image also has two blue traced dots marking the start and end of a centimeter, so that a metric can be established. The format of this data makes it ideal for better surface reconstruction because of the possibility to accurately compute a metric and obtain a very dense sample point set.

We developed a simple java program that employs color threshold and blob detection techniques found in the OPENCV computer vision library. This program is able to extract around 3000 points form a 7-slice photo and around 450 points from the overhead photo. The extracted points have a counter clock wise orientation and start for the lowest point in each detected object.

The explicit approach to surface reconstruction consists in approximating a given shape as a collection of simple geometric objects such as curves and polygons. We are given initially a finite set of points P taken from that shape. The objective is to approximate the shape with simple objects.

There are many ways of realizing this goal. Popular explicit representations include parametric surfaces and triangulated surfaces. We focused our work on triangulation, representing the shape as a collection of triangles. This representation has the advantage of counting with robust implementations of the geometric tools needed and a convenient representation for measuring the object. Due to this representation the reconstructed surface is a linear piecewise manifold. Information between sample points is linearly interpolated. This shape has similar topological and geometrical properties to a real placenta: a closed connected shape without holes. In this embodiment we used these reconstruction approaches: Power Crust algorithm; Special Triangulation; Delaunay Triangulation; Voronoi Diagram; Medial Axis.

We use a fast tagging algorithm to obtain a good initial surface. The local level set algorithm makes it possible to apply level set method in three dimensional image reconstructions within a reasonable amount of time. Instead of updating the level set function on the whole grid, we only update the level set function on and near the boundary. Here we first present the outline of our algorithm, and in the following sections we explain the detailed implementations.

The steps of the main algorithm, Local Level Set Method, are as follows: (a) First, compute the distance function d of the sample points on the whole grid using the Fast Sweeping Method; (b) Compute the gradient of the distance function. (c) Create the level set function with the zero iso-contour enclosing all the sample points. In this embodiment, our initial guess is a box which captures the overall shape of the placenta; (d) Use the Fast Tagging Algorithm to obtain a good initial guess; (e) Reinitialize using Equation to render the initial guess a signed distance function. Then, solve for the level set locally.

Fast Sweeping Method.

First construct a distance function at each grid point associated with the sample points. For our small data sets, this could be done timely by “brute force”, i.e. computing the distances from a grid point to each sample point and choosing the smallest one. However, the surface so reconstructed has deep troughs at each slice on the side of the chorionic plane.

The sweeping method solves the Eikonal Equation to its steady state. The sweeping method applies the Gauss-Seidel Iterations with alternating orders to solve for the stationary state of the equation above. In the three dimensional case, we sweep the grid eight times along each diagonal. We first found that this method was 50% to 80% slower than the brute force method for data sets of 300-800 points. However, we also were surprised to observe a smoothing effect of this method: the deep “cuts” at slices on the chorionic plane disappeared.

Fast Tagging Algorithm.

Finding a good initial guess is the next step to ensuring the accuracy of the surface reconstruction based on 3D level set method. The Fast Tagging Algorithm is designed to reduce the computational expenses and numerical errors of updating the level set function. The Tagging Algorithm produces a coarse surface at an adjustable distance to the sample points without introducing additional numerical errors. This feature allows the tracking distorted shapes more accurately. After the tagging algorithm produces a good initial guess, only a few iterations are required to smooth the surface.

The steps of the Fast Tagging Algorithm are as follows: (a) The sample points are used to produce a first guess. In this application, the zero iso-surface is a box enclosing all the sample points; (b) Points that are on the zero iso-surface and have an interior neighbor are labeled as temporary boundary; (c) Points labeled as temporary boundary are sorted and the point with the largest distance is selected; (d) There are two possible cases: first, if this point has an interior neighbor that has a larger distance, then set the former to permanent boundary; second, if the first case is untrue, then eliminate this point from the vector of temporary boundary and put in its interior neighbors; (e) Repeat the previous two steps until the largest distance is less than a tolerance. Then set every point labeled as temporary boundary to permanent boundary.

Re-Initialization.

As the level set function evolves, it will generally drift away from a signed distance function. Numerical errors accumulate where the local gradient increases or decreases substantially. Therefore, it is necessary to re-initialize the level set, for example using the re-initialization equation taught in: M. Sussman, P. Smereka, and S. Osher Journal of Computational Physics, 114(1):146-159, 1994. A level set approach for computing solutions to incompressible two-phase flow, was found to be effective. Solving this equation to its steady state renders the level set function a signed distance function. After the Fast Tagging Algorithm produces a good initial guess the re-initialization is applied on the grid to smooth out the level set function.

Partial Differential Equation-Based Fast Local Level Set Method.

If the level set function is updated globally, the computational expenses are O(n3) in three dimensions, where n is the length of the grid. The PDE-Based Fast Local Level Set Method by Peng et al. (J. Computational Physics, 1999, 155:410-438) confines the region of computations to a narrow band on and near the zero iso-contour of the level set function. This technique reduces the computational expenses to O(N), where N is the number of points on the implicitly reconstructed surface.

In another embodiment this algorithm was implemented for our surface reconstruction. We first define the inner tube. Within this tube, the level set function will be updated. We also define the outer tube. Inside this tube the level set function will be re-initialized after each update. After each update and re-initialization, the tube will be expanded according to the following rule: search for the points that are inside the tube and have a neighbor whose distance function value is less than a certain threshold; then add this neighbor to the tube.

Convergence and Accuracy.

We find that the main shape and important surface features are accurately reconstructed by comparing the reconstruction with the original image in FIGS. 5C and 5D. The total energy gives another measure of the accuracy. It has been demonstrated that surface energy is minimized as the solutions, therefore the iso-surface, converge. Accuracy can therefore be achieved by approaching the minimum energy as close as possible and still remaining outside the surface. The tagging algorithm ensures that the points on the initial guess are approximately equidistant to the set of sample points.

Therefore it is only necessary to update the PDE based level set function a number of times, as each iteration advances the zero iso-surface approximately the same distance. Over-updating, on the other hand, tends to push the surface inside the sample points and the surface will collapse into the empty set. The tagging algorithm partially resolves this problem by tracking the topology of the surface closely. We use the simple and expensive way of using larger grid to raise accuracy. Since the steps of updating the level set function using the convection model or energy minimizing model are fixed, and the Fast Tagging Method pushes the zero iso-contour to the sample points at the same threshold, the number of points outside the surface and their relative positions are the same. In the meantime, the spatial step size is smaller in a larger grid, therefore the exterior points are closer to the sample points.

Volume, Surface Area and their Ratio.

Explicit Method.

Calculating the Surface Area (SA) from the reconstructed shape involves summing all the boundary triangles created by the special triangulation. Calculating volume (V) is accomplished by filling the space created by the boundary triangles and the planes that cut adjacent slices. This space is filled with simple polyhedra for which simple volume formulas are known. Our method creates an inner pyramid in between slices. This is using a polygonal slice as the base and the adjacent slice centroid as the apex. Each tetrahedron created has a triangle lying on a pyramid. It also contains a boundary triangle that shares an edge with the pyramid triangle. These two triangles have 4 points which are sufficient for defining a tetrahedron. Since this is done for each pyramid triangle, i.e. for each side of the polygonal base, then it is guaranteed this procedure will fill the remaining space. The volume will be the sum of all the tetrahedra and pyramids created in this process.

Implicit Method.

Calculating the volume and surface area in the implicit method is done by integrating the surface.

Chorionic Surface Vascular Branching.

Chorionic surface vascular branching is laid down by the middle of the second trimester, and the principal branches off the umbilical cord insertion reflect the state of the primordial placenta shortly after the onset of the beating fetal heart. As such, the number of such vessels, the number of branch points, inter-branching intervals, and the total vascular length are measured to quantify aspects of endothelial proliferation and gene events relevant to placental branching early in gestation. At the same to early gestational ages, fetal viscera such as lung, kidney, and pancreas are also using the same gene families, and the same molecular signals and cascades to induce growth and branching growth.

Image Enhancement Using Polarized Light.

The detail in a digital image of the gross placenta is often obscured by glare from ambient lighting on the moist chorionic surface of the placenta. The glare is often bad enough to make automated image analysis impossible since image segmentation algorithms misclassify the pixels in the sections of the image subject to glare.

To eliminate the glare, a camera stand was constructed so that both the light source and the camera were fitted with circular polarizing filters. Both filters were rotated to minimize glare which was essentially eliminated. A second light source was added to the camera stand to eliminate shadows. It was also fitted with a circular polarizing filter. The two polarizing filters were rotated so that their light was aligned in the same direction. The lights were positioned so that shadows were eliminated and glare was also found to be eliminated. Plane or linear polarizing filters can also be used to remove glare.

Automated Vasculature Extraction from Placenta Images.

Recent research in perinatal pathology argues that analyzing properties of the placenta may reveal important information on how certain diseases progress. One important property is the structure of the placental blood vessels, which supply a fetus with all of its oxygen and nutrition. An essential step in the analysis of the vascular network pattern is the extraction of the blood vessels, which has only been done manually through a costly and time-consuming process. There is no existing method to automatically detect placental blood vessels; in addition, the large variation in the shape, color, and texture of the placenta makes it difficult to apply standard edge-detection algorithms. We describe a method to automatically detect and extract blood vessels from a given image by using image processing techniques and neural networks. We evaluate several local features for every pixel, such as intensity, gradient, and variance, in addition to a novel modification to an existing road detector. Pixels belonging to blood vessel regions have recognizable responses; hence, we use an artificial neural network to identify the pattern of blood vessels. A set of images where blood vessels are manually highlighted is used to train the network. We then apply the neural network to recognize blood vessels in new images. The network is effective in capturing the most prominent vascular structures of the placenta.

Pre-Processing.

Before being analyzed, placental images are preprocessed to ultimately improve the performance of subsequent algorithms. Without the preprocessing steps, the samples are too difficult to properly analyze because the contrast between the tissues of the vascular network and the surrounding tissue is not sufficient. Thus, we must alter the specimen to improve the contrast between the vascular structures and the surrounding tissue. To achieve this improvement in contrast, we insert two catheters into the umbilical vein and an umbilical artery, and tie securely with suture. Working first with the umbilical vein, the placental fetal vasculature is heparin flushed and perfused with a 1% agarose 30% barium sulfate solution. We then apply a directional multiscale mathematical framework based on the combination of shearlets and Laplacian Eigenmaps. Shearlets, a method of the wavelet family, efficiently represent images with directional elements. Laplacian eigenmaps, a non-linear manifold learning technique highlights the curvilinear features remaining after applying shearlets. Our method is capable of extracting arteries and veins of various sizes and learning directional and anisotropic variability exhibited through the tortuous vasculature network. We then extract the placenta by applying a threshold and some morphological operations on the green channel. We then crop the image, thereby making future calculations more efficient.

Many images feature large patches of glare, so an in-painting approach is used. First, bright spots are identified as those pixels with intensities above a pre-determined threshold, which we take to be 80% of the maximum intensity. Second, a top-hat filter is applied, and additional thresholding then accurately identifies appropriate glare regions. Third, the regions are dilated by several pixels in order to place the region boundaries on pixels unaffected by glare. Finally, solving Laplace's equation fills in the regions, which produces satisfactory results. We found that performing glare removal prior to the cropping procedure is preferable, as otherwise some glare regions could be unintentionally cropped.

Alternately, using polarized light to illuminate the placenta and capturing the image using a polarized filter will remove glare.

Features.

We use a neural-net approach to extract vascular features. This meant that numerous features would be computed for a placenta and then later fed to a neural network to detect vessels. Some features that were computed on placenta images are described in the following subsection. Other features include variance, curvature, eigen-values of the 2nd moment matrix, gradient magnitude, and gradient orientation.

Line Detectors.

In the green channel individual vessels show little variance in intensity after glare has been removed. Hence, we focused on those that could detect thick, uniform, curvilinear structures. We implemented several conventional line detectors, such as Stager's line detector, a phase-coded detector, and a slightly modified wide-line detector, to be used as additional features for the neural network.

Stager's Detector.

As provided, the Steger detector only gives a response at the center and on the edge of a thick line. Hence, to make this output more appropriate as a feature, these lines were filled in. At each pixel along the center of a line, pixels along the normals (for the appropriate width) were assigned a value of the line's “response,” or the second-derivative of the line at that point, as described by Steger. This yielded results surprisingly similar to the wide-line detector; the two methods largely agreed on the larger vessels, but differed more in the noisier, vessel-free regions of the placenta.

Modified Road Detection

A novel modification to an existing road-detection technique made it a much more suitable method for detecting vessels. Porikli uses a directional line filter to look for elongated rectangular regions in-between two homogeneous regions of different intensity levels on either side. This filter is somewhat limiting because it can only detect lines with a maximum thickness of five or six pixels, whereas blood vessels can be much thicker.

We multiply Porikli's directional line filter by a Gaussian function in order to allow the filter to identify wider structures, with more weight towards the center. We found that our enhanced road detection method was superior to the other line detectors when used on placental images. However, in another embodiment we additionally used the neural-net approach to resolve the output from all of the features above, particularly the line detectors, while minimizing false-positives in the final result.

Neural Network Training.

Manually tracing a vasculature network is subject to human interpretation. This imprecision can affect the accuracy of the neural network output. To reduce the impact of these outliers, we added an option to transform the binary traced data to grayscale by convolving it with a Gaussian kernel, thereby giving greater weight to regions that had been traced while still allowing positive responses outside the traced areas. We also used mean-absolute error instead of mean-squared error when evaluating a network's performance, as it is more robust to outliers in the training data. To determine the optimal combination of the features, numerous neural networks were trained with all possible combinations of 3-or-more features from all available features. This exhaustive search of the feature space was necessary because the processes of neural network training are not sufficient to determine which features are unneeded. In addition, various parameters for the networks, such as the performance function (mean-squared error or mean-absolute error), the number of hidden nodes (5 or 15), how to normalize features, and whether to apply a Gaussian blur to training data can also be applied.

Post-Processing.

Our neural networks largely produced soft classifications of blood vessels, so further processing of these results was necessary. Grayscale neural-net outputs were thresholded to obtain a binary classification. These black-and-white images were then filtered for size; components smaller than usually 400 pixels were discarded as noise.

Results.

A result of our method is shown in FIG. 6. While not perfect, the neural network does identify many prominent vessels. We note that the width of the detected vessels is more accurate than in the manual tracing. We found that, in general, nets that used the mean-absolute error for their performance function performed slightly better than those that used mean-squared error. Blurring training data to reduce the impact of outliers had little effect on performance. Somewhat surprisingly, networks with only five hidden nodes performed better than networks with 15 or more hidden nodes; they were also faster to train and simulate.

Currently, inputs to our neural networks are features computed for individual pixels. To make the results of these networks more context-aware, we could feed the network features computed at the pixel in question as well as the features for all neighboring pixels. Other learning methods such as k-nearest neighbors can also be used. In another embodiment, a C implementation of the wide-line detector would improve speed. In a further embodiment, Steger's detector could also be used to more easily distinguish between arteries and veins.

Image segmentation: Histologic placental features—Current diagnostic types (acute inflammation, chronic inflammation and vascular pathology).

While histopathologic identification of specific features is the prior art method for diagnosis of inflammation and hypoxia, the diagnosis of these processes, each with well-characterized fetal, neonatal and potentially lifelong impacts, remains problematic. Interobserver reliability, even with a test set of 20 slides, 14 of which had lesions, yielded reliability coefficients that were primarily only “fair”. Furthermore, “consensus” was the gold standard, not a specific maternal, fetal or neonatal outcome, nor was an objective morphometric quantification provided for such items as “neutrophil count” or “syncytial knotting”. The digitization of images and, more recently, entire histology slides, has moved each of these into the realm of “data”, accessible (as pixels) to mathematical manipulation.

Stained histology slides of placental tissue produce images with highly concentrated color spectra, making these images strong candidates for the use of automatic image segmentation and object classification algorithms.

Image Segmentation: Villous Branching Structure.

While at least some methods for histopathologic identification of inflammation and hypoxia exist, the prior art has no standard method for the analysis of placental branching architecture. Advanced mathematical techniques are well suited for the quantitative analysis of placental branching architecture, and the quantities so extracted can be entered into models to study their contributions to causal pathways of fetal disease. However, placental arborized structure, as measured after delivery, reflects the effects of the underlying maternal uteroplacental environment. That environment is not directly observable (hence “latent”) but it causes the observed placental arborized structure. Empirically, then, measures of placental arborized structure and the maternal uteroplacental environment should be correlated, and the relationships among a set(s) of measured histological parameters related to placental arborized structure can be examined. Examples of histological parameters include, but are not limited to, villous numbers, villous areas, villous perimeters, trophoblast features including thickness, vascular features including medial characteristics, luminal perimeter and location within the villus (central versus subjacent to the trophoblast epithelium).

Further examples of histological parameters include, but are not limited to, syncytial knots (e.g., dark blue cluster of round objects); perivillious fibrin/fibrinoid (e.g., pink and devoid of nuclei the size of normal villous Syncytiotrophoblast, stroma and endothelial cells); cytotrophoblast proliferation (useful, for example, to distinguish “old” PVF from recent PVF; e.g., nuclei of the size of cytotrophoblast cells which should be distinct from villous stromal and other nuclei, found in PVF); and stromal cellularity (e.g., nuclear number within each distinct villus or maybe better nuclear area per villous area). Such histopathology parameters can be detected in, for example, H&E slides. Still further examples of histological parameters include, but are not limited to, syncytiotrophoblast; endothelium (useful, for example, to verify H&E stained algorithms); macrophages (e.g., 40-60% of villous stromal cells are immunocompetent macrophages); and anchoring and endovascular trophoblast (useful, for example, to shifts focus from villous arborization to the placental remodeling of the implantation site, which moves analysis into earlier times of gestation, and ultrasonographic correlation). Such histopathology parameters can be detected in, for example, immunohistochemical (IHC) stained or in situ PCR slides for cell proliferation, cell activation, cell death and gene expression.

The methods described herein can be employed to reliably diagnose placental villous branching patterns that, to date, cannot be reliably diagnosed including, but not limited to, the 6 paradigm branching patterns as elaborated by Kaufmann (Normal preterm placenta (defined as prevalence of immature intermediate and mesenchymal villi, complete absence of mature intermediate and terminal villi, poorly matured stem villi), Immature placenta at term (defined as prevalence of mature intermediate and stem villi, paucity of immature intermediate and terminal villi), Normal term placenta (defined as a generally even distribution of all types of villi), Preterm preeclampsia (the pathology classic for maternal vascular pathology, defined as poorly branched, extremely tiny, filiform terminal villi and because of paucity of terminal branching, an unusually wide intervillous space), Term preeclampsia (defined as a generally even distribution of all types of villi, with terminal villi generally being enlarged and highly branched), and two cases of malformed villi with normal numerical mixture of villous types).

The methods described herein can also be employed, for example, to: diagnose time of onset of placental pathology (through “branching tree” analysis); quantify the effect of abnormal placental growth on the fetus; reliably diagnose fetal growth restriction including abnormal growth within the “normal” birth weight range; diagnose which cases of maternal diseases (such as diabetes, preeclampsia) affect the growth of the placenta and/or growth of the baby and which do not; document treatment efficacy and treatment failure in patients treated in a subsequent pregnancy after a pregnancy loss or serious complication; and diagnose which pregnancies following IVF/ART have abnormal placental growth and which do not.

Abnormal placental branching could be associated with childhood (and potentially lifelong) abnormal function of organs that are undergoing branching growth at the same time as the placenta. Thus, the methods described herein can also be employed to diagnose risk for abnormal neurodevelopmental outcome (analysis of neuron branching growth); risk of insulin resistance and abnormal glucose metabolism, obesity and diabetes (analysis of pancreatic branching growth); hypertension (analysis of branching growth of the cardiovascular system); reduced renal reserve/risk of hypertension, renal dysfunction (analysis of kidney branching growth).

Furthermore, by building language algorithms for identifying pathology lesions that are currently recognized correlates with maternal and fetal/neonatal pathologies, one can provide an automated and reliable diagnostic service to a field with few dedicated practitioners and with a large need for such services (e.g., community hospitals, academic centers without a dedicated practitioner, in medicolegal field/risk management with “competing experts”) such as histology features that diagnose, for example, acute intraamniotic infections, chronic placental inflammation, maternal uteroplacental vascular pathology and fetal-placental vascular pathology. Thus is provided methods to diagnose the mechanistic cause and the time of onset of pathologies that create ill newborns or stillborn fetuses. More generally, the methods described herein allow identification of the time of onset of the histology features described herein and quantify their total effect on the fetus (via their effects on placental growth globally).

Registration and Reconstruction of Branching Architecture.

Histology slides are two-dimensional slices from a three-dimensional placental volume. Image registration is the process of transforming the different sets of data into one coordinate system. Registration is necessary in order to be able to compare or integrate the data obtained from different measurements. Using image registration many slices can be combined to form a sub-volume of the three-dimensional structure. Two powerful registration methods can be combined in placental registration: Area based image registration algorithms and related methodology look at the structure of the image via correlation metrics, Fourier properties and other means of structural analysis; feature based methods, instead of looking at the overall structure of images, map to image features: lines, curves, points, line intersections, boundaries, etc. Combining segmentation techniques with registration, parts of the villous tree internal to the placental volume may be extracted and studied. Using these methods to segment histology images as well as images of the chorionic surface produces the geometric structure of several parts of the placental villous tree.

Very simple morphological methods can be applied to skeletonize the geometry and construct a representation of the tree as an embedded (i.e. geometric) graph. Graph theoretic techniques are applicable to analyzing the anatomical structure of the villous tree. Metrics can be designed from both the geometry and topology of the villous tree. For example, topological metrics count how many leaves are on each tree or how many levels of branching there are at each leaf, while geometric metrics examine how far traveling between two points in the tree compares to traveling in a straight line across the chorionic plate. Additionally, combined metrics, i.e., metrics that consider both the geometry and topology of the tree, can be used. One example is to measure the length between branch points at each level of branching. During the skeletonization process, some information may be lost (such as the thickness of the vessels), but some of this information can be retained by assigning weights to the edges of the constructed graph and viewing the resulting structure as a flow network or as a self-organizing map, a method that has been useful in dimension reduction.

Validation of placental measures and models: Placental function depends on placental architecture.

Another embodiment described herein are based at least in part upon application of the discovery that placental growth scales to fetal growth to the three-quarter power, essentially consistent with scaling typical of fractal transport networks, and that altered placental shapes have scaling factors that deviate from the three-quarter rule, consistent with altered placental shapes reflecting altered underlying placental vascular fractal networks.

Modeling placental function and growth are accomplished separately. As stated above the primary function of the placenta is maternal-fetal transfer, therefore models of the vascular tree can be produced that optimize the transport function. Data analysis tools are then applied to compare specific villous trees with trees generated by this model to determine how far the given placental tree is from the “optimal” tree.

Optimal transport can be used to validate our measurement methods; in other words, the villous (and by extension, the underlying vascular) architectures we reconstruct are directly related to the estimated transport function of the placenta. Diffusion limited aggregation (DLA) is a stochastic process that can be applied to dynamically model angiogenesis in the placenta, thereby modeling placental growth. DLA has been used to model retinal and tumor angiogenesis. Dynamic models of placental growth can be used to investigate the effect that environmental changes at different stages of the growth cycle have on the resulting vascular structure. DLA may be particular useful in maintaining a reasonably good agreement between the observed scaling exponent, approximately 0.75, which we have found to be appropriate for describing the relationship between placental structure and placental function.

Dynamical modeling of vascular trees is a new technique, developed by M. Yampolsky and his team at the University of Toronto. There have been prior art efforts to model the complex architecture of a vascular tree. They have been based on selecting certain geometric constraints for the tree, such as the number of branches at each vertex, and the branching ratios; and then optimizing the tree to fill the spatial shape of the organ. This approach is static in its nature, and does not give clues to the temporal development of the vasculature, and thus is not suitable for determining how growth pathologies affect the development of a placental vascular tree and the geometric shape of the placenta. The dynamical growth process in this invention is based on sprouting angiogenesis which is the mechanism of growth at the tips of the vessels. With each time increment, the model vascular tree is randomly grown at one of its extremities, with a single parameter controlling the density of the branching. This random growth process is known as DLA. Applying a “hit” to the parameter of the model at a specific moment of time we influence the development of a particular level of the vasculature.

The model successfully reproduces the variability of shapes of pathological placentas. Quantitatively, the deformations in the model trees will be described by the changes in the average number, length, and thickness of branches. This makes it possible to introduce measures of the deviation from the normal, and to search for markers corresponding to the specific changes in the vascular structure. Another approach to measuring the deviation from the normal relies on measuring the optimality of the branching architecture. Possible conditions of optimality reflect the efficiency with which the blood flow is delivered to the tissues. They translate into an optimal local geometry of the vascular tree. Under the assumption that a normal vasculature is close to optimal, the deviation from normal growth can be expected to induce a measurable decrease in optimality.

In summary, the tools we apply to placental measurement fully characterize the histopathology and the architecture of a fetal organ the growth of which depends upon pathways critical to the genesis of autism and other childhood and adult morbidity risks. Finally optimal transport analysis and DLA confirm that our measures and reconstructions are valid and relevant to placental function and fetal-placental physiology.

A preferred embodiment of the present invention is a method of predicting the potential for manifestation of various medical conditions by analyzing the placenta. This is accomplished by first determining the need for early monitoring, intervention or potential treatment for medical conditions likely to manifest as a child grows older. Once the need is recognized, a physician begins by selecting and identifying a sample of the placenta to analyze. Ultimately, the analysis will be performed by computer applied mathematical algorithms, but a key to accurate results is the preparation of the samples which will supply the data to the computer and thus the data for the algorithms. A physician continues by determining the proper capturing device to obtain a three-dimensional image of the sample. Once a decision is made, the physician commences preparing the placental sample to be analyzed. The preparation of the sample can take many forms, some conventional, and some novel. For example, barium can be added to the placental sample to greatly improve the contrast between the vascular structure and the surrounding tissue. While the vascular structure and surrounding tissue are typically maroon in color and difficult to separate, addition of the barium turns the vascular structure a white-ish color, thereby greatly increasing the contrast with the surrounding tissue and enabling the physician to better analyze the sample.

Further, the physician continues by obtaining a three-dimensional digital image of the chorionic surface of the placental sample by use of the selected capturing device. Once the digital image is captured in a computing device, the physician must manually correct for errors inherent in digital image acquisition. The physician, not the computer, selects the corrected digital image data to input into the computer for analysis. Once the data is in the computer, the computer performs an analysis on the corrected digital image data using one or more algorithms to determine the vascular structure of the placenta. When the vascular structure of the placenta is determined, the vascular structure can be analyzed to determine the potential for manifestation of various medical conditions.

It is further contemplated that the disclosed method can be modified where the digital image is obtained by configuring the selected capturing device to use polarized light or a polarized filter. Additionally, as indicated, the preparing of the placental sample can include staining or treating said placental sample to increase transparency.

It is still further contemplated that this method can be further modified by using one or more dedicated mathematical algorithms including image segmentation, branching metrics, fourier analysis, or other graph or network metrics that are used to assess the timing of an event or stress including infection to the developing placenta or fetus. Further, analyzing the results of the determined timing of an event or stress can be achieved by combining the placental measures into a dedicated predictive model and using the model to assess future health risks to a patient.

It should be appreciated by those skilled in the art that the sample to be analyzed can be selected from the group comprising the entire placenta, portions of the placenta, the placental shape, the umbilical cord, or the membranes. Also, the proper capturing device to obtain the highest quality three-dimensional image of the sample can be selected from the group consisting of film, a digital camera, a 3D scanner, confocal microscopy, or 3D printing in conjunction with a microscope having a resolution capable of magnification of 20-40× power.

A still further refinement to the instant embodiment can include cropping the three-dimensional image to make the algorithm calculations more accurate and removing glare by identifying pixels with intensities above a pre-determined threshold and excluding those pixels from the calculations. Still further one can practice this method by analyzing identified structures to determine whether the structures are connected or disjointed and resetting the digital data collected by the image to reflect the proper structure as determined by the physician. In addition, the physician can apply a top hat filter and dilate the region by several pixels, and can use Laplace's equation to fill in the regions. As stated, this method can be used in analyzing the data to quantify the effect of abnormal placental growth on the fetus and predicting abnormal function of organs that are undergoing branching growth at the same time as the placenta.

Data Reduction.

Computer-assisted image analysis minimizes measurement error of individual histology items and while each item may be reliable, the complexity of placental arborized structure requires measuring so many histology items that data reduction is required before examination of the predictive effects of different patterns of placental arborized structure. Various strategies of data reduction can be employed.

Reliability and Validity of Measures.

While data might be reduced to a parsimonious set of factors, the factors may not reliably measure what they are intended to measure. In evaluating reliability of quantification of individual histology items and of the EFA/CFA factors, several reliability tests can be used. For example, multiple tissue samples from one placenta are multiple “tests” of that placental structure. As another example, “test-retest” reliability can assess reliability of histology item quantification and also the extent to which placental structure (reflected in factors as combinations of related histology item scores) is stable across multiple tissue “tests”.

The analysis methods may generate a large number of quantified variables relating to aspects of histology items, many of which are intercorrelated. When many potentially parallel histology items are present, histology items can be split in two to test “alternate forms” reliability. This approach can test whether items missing could be substituted with other items and the overall measures remain reliable. Such flexibility allows for tools to be robust to inevitable variability in tissue sampling techniques (that may result in missing histology items) when employed on large populations. Generalizability is the extent to which the measurement process is equivalent across dimensions. Potential sources of variation include, for example, gestational age at delivery, maternal disease states (e.g., preeclampsia, diabetes), and exposures (e.g., maternal smoking). Procedures described herein are sufficient to test whether the measures are consistent across strata.

To determine the associations between placental structure and childhood outcomes, structural equation modeling (SEM) can be employed. SEM explicitly models factors as mirrors of latent variables to test the relationships among factors, covariates and outcomes. MPlus (Muthen and Muthen Mplus: Los Angeles, 2006) is an especially flexible SEM tool that accommodates categorical and continuous latent variables, and latent class analysis. SEM is a linear modeling approach and, as such, provides for modeling factors that are linear combinations of histology items.

Diffusion and Diffusion Screening in the Placental Villi.

The mature placenta is a complex arborized vascular bed extending from the umbilical arteries to the chorionic surface vessels, to the fetal stem vessels and ultimately to the capillary beds of the terminal villi, the anatomical sites of all oxygen and nutrient exchange between the mother and the fetus. The capillary beds drain into a venous system that parallels the arterial tree, ultimately draining into chorionic surface veins and the umbilical vein that carries blood to the fetus.

The fetal blood is contained in the fetal capillaries of the chorionic villi, and the maternal blood flows in the intervillous space. The placenta can therefore be conceptualized as an exchange unit. The respiratory functions of the placenta make it similar to lungs in terms of exchange of oxygen and carbon dioxide. Respiratory transfer from the mother, across the placenta, to the fetus occurs in three steps: first, the maternal blood brings oxygen to the intervillous space which bathes the fetal chorionic villi; second, oxygen permeates across the villus surface and diffuses inside the villusstroma toward the fetal capillaries; third, oxygen is transported to the fetus via fetal blood. The explicit separation of the transport in these three steps is not only physically justified, but it allows one to consider each step separately, the output data of one step serving as the input data for the next step.

As the villous and bronchial structures are both branched, it is natural to expect analogies between the fetal blood flow in the fetal capillary tree (the third step of the placental function) and the air flow in the bronchial tree. The first two steps of the placental function also have many common physical features with the oxygen transport in the lungs, although the maternal intervillous space has no true vascular structure and merely forms a pool around the villi. In this analogy “screening” effects are considered along with their potential relation to diseases. This consideration relies on a comparative analysis of high-resolution two-dimensional (2d) cuts of normal and pathological placentas.

We first focus on the step in which oxygen dissolved in the maternal blood is brought by flow into the maternal intervillous space to access the placental villi. Maternal blood flows through . . . 100-150 uteroplacental arteries and enters the intervillous space at a high flow rate but a very low pressure (10-15 mm Hg). This oxygen rich maternal blood bathes the villi, containing capillaries carrying poorly oxygenated fetal blood. Driven by this difference in partial pressures, oxygen permeates from the intervillous space to the villi across the villous surfaces, the maternal and fetal circulations remaining separate. In turn, carbon dioxide permeates the villous surface in the opposite direction, from the villi to the intervillous space. After oxygen and carbon dioxide are exchanged, the oxygen-depleted maternal uteroplacental arterial blood drains out of the intervillous space and returns to the maternal circulation via the endometrial veins.

The placental villous branches (with, at their tips, the terminal villi) present geometric obstacles to maternal intervillous flow; maternal intervillous flow rate declines from the basal to the chorionic plate. From the perspective of maternal perfusion, intervillous blood flow will access individual terminal villi at different flow rates, greater for terminal villi closer to the maternal basal plate, and slower for terminal villi near the chorionic plate (the fetal surface of the placenta). This is analogous to what happens in the bronchial tree of the lungs, in which fresh air is inhaled through the mouth at relatively high velocity and then substantially slowed down as it moves into the distal bronchioles (with their greater total cross-section area).

Normal Versus Abnormal (Pathological) Placentas.

Various processes (maternal diseases, environmental exposures, etc.) can lead to abnormal growth of the placental villous tree. Abnormal development of the placental villous tree (over growth or sparse branching) makes either or both maternal uteroplacental blood flow around the villi and fetoplacental blood flow within the villi less efficient; both contribute to abnormal placental-fetal transport. Transport is more efficient when all the terminal villous surfaces are equally accessible to the maternal uteroplacental intervillous blood. However, an abnormally grown placenta with an increased number and/or size of villi (e.g., diabetic placentas) may have “crowded” villi. Villi in too close proximity may “shield” each other from the maternal perfusion and limit their function. As a result of over crowding, maternal blood cannot flow easily around these terminal villi, and transfer of oxygen from the maternal circulation across the villus surface is substantially reduced. Dense packing of the villi makes them more “shielded” (or “screened”) to the flow of the maternal uteroplacental intervillous blood. Conversely, too sparse villous arborization results in maternal uteroplacental intervillous blood flow that cannot adequately access terminal villi; maternal blood may flow into and out of the intervillous space without encountering villi and transferring any oxygen, another type of inefficiency.

From this functional point of view, the difference between normal and pathological placentas resembles the difference in functioning of the lung acinus at exercise and at rest. In the normal placenta, all the terminal villi are accessed more or less equally around their entire perimeter by the maternal uteroplacental intervillous blood (as the alveolar membrane is accessed by oxygen at exercise). In pathologically “overgrown” placentas, the intervillous space is crowded with villi so that only a fraction of the terminal villous surfaces can be accessed (only a part of the alveolar membrane near the acinus entrance is accessed at rest). The effect of diffusion screening is expected to play a crucial role in placental transport, especially in abnormal placentas (e.g., for diabetic women). Although the lung-placenta analogy is instructive, there is a significant difference between the lungs and the placenta. In the placenta, the maximal accommodations to blood flow are part of normal pregnancy, e.g., maternal heart rate increases, total peripheral resistance drops, plasma volume increases resulting in a dilutional anemia that reduces shear stress, as well as hormonally dependent increases in endometrial flow. Thus, there are no more physiologic adaptations that can be made to increase intervillous perfusion. In contrast, one can increase alveolar aeration by increasing rate and depth of inhalation in order to “switch” between reduced efficiency of the lungs at rest and their “full” efficiency at exercise.

Having described the invention in detail, it will be apparent that modifications, variations, and equivalent embodiments are possible without departing from the scope of the invention defined in the appended claims. Furthermore, it should be appreciated that all examples in the present disclosure are provided as non-limiting examples. Further, it is the intent of the inventors to claim all novel aspects of the method, including any equivalents, all of which are intended to be encompassed within the claims.

### REFERENCES CITED

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention. It shall be understood that any method described in an example may or may not have been actually performed, or any composition described in an example may or may not have been actually been formed, regardless of verb tense used.

### Example 1: Extracting Neutrophils

A placental sample was taken from the placental membranes of a term fetus. A slice of the tissues was prepared using the standard procedure for preparing a histology slide. The tissue was fixed in formalin, de-hydrated, embedded in a paraffin block, a thin slice was microtomed from the block, and affixed to a glass slide. The slide was placed in an Aperio T3 slide digitizer and the image produced at a magnification of 20×. The digitized image was processed using the SFCM algorithm. The parameters of the algorithm were set to extract the color differences of the neutrophils. Both the original image and the extracted image are shown in FIG. 1. The extracted image separates the neutrophils from the remainder of the image. The high incidence of neutrophils indicates a higher risk that this child will develop Cerebral Palsy.

### Example 2: Extracting Tissue Edema

A placental sample was taken from the umbilical cord of a term fetus. A slice of the tissues was prepared using the standard procedure for preparing a histology slide. The tissue was fixed in formalin, de-hydrated, embedded in a paraffin block, a thin slice was microtomed from the block, and affixed to a glass slide. The slide was placed in an Aperio T3 slide digitizer and the image produced at a magnification of 20×. The digitized image was processed using the SFCM algorithm. The parameters of the algorithm were set to extract the clear areas that characterize edema. Both the original image and the extracted image are shown in FIG. 2. The extracted image separates the areas of edema from the remainder of the image. The presence and extent of edema indicates an abnormal tissue function associated with poor neurodevelopmental outcome.

### Example 3: Extracting Connective Tissue

A placental sample was taken from the placental membranes of a term fetus. A slice of the tissues was prepared using the standard procedure for preparing a histology slide. The tissue was fixed in formalin, de-hydrated, embedded in a paraffin block, a thin slice was microtomed from the block, and affixed to a glass slide. The slide was placed in an Aperio T3 slide digitizer and the image produced at a magnification of 20×. The digitized image was processed using the SFCM algorithm. The parameters of the algorithm were set to extract the grayscale intensity differences of the characterize connective tissues. Both the original image and the extracted image are shown in FIG. 3. The extracted image separates the connective tissues from the remainder of the image. The damage seen in the connective tissues of the fetal placenta reflects breakdown of those tissues from digestive enzymes which are associated with an increased risk of damage to the child's heart, lungs, and brain.

### Example 4: Placental Shape as Reflective of Placental Function as a Fractal Network

Subjects were a subset of the National Collaborative Perinatal Project (NCPP). Details of the study have been described elsewhere [19, 20]. Briefly, from 1959 to 1965, women who attended prenatal care at 12 hospitals were invited to participate in the observational, prospective study. At entry, detailed demographic, socioeconomic and behavioral information was collected by in-person interview. A medical history, physical examination and blood sample were also obtained. In the following prenatal visits, women were repeatedly interviewed and physical findings were recorded. During labor and delivery, placental gross morphology was examined and samples were collected for histologic examination. The children were followed up to seven years of age. Placental gross measures included placental disk shape, relative centrality of the umbilical cord insertion, estimated chorionic plate area, disk eccentricity, placental_disk thickness, placental weight, and umbilical cord length, measured according to a standard protocol. Gestational age was calculated based on the last menstrual period in rounded weeks. Among 41,970 women who gave the first or only singleton live birth, 36,017 contributed placenta data. The analytic sample was restricted to those with complete data on the six placental gross measures, placental weight and birth weight, of gestational ages >=34 weeks (younger infants having been unlikely to survive) and less than 43 completed weeks (given that gestations were assigned implausible gestational lengths up to 54 weeks, N=24,061).

Chorionic disk shape coding was based on the gross examination of the delivered placenta. Shapes included round-to-oval, and a variety of atypical shapes (e.g., bipartite, tripartite, succenturiate, membranous, crescent or “irregular”). Only 926 (3.8 percent) were labeled as one of the 6 categories of shape other than round-to-oval. For this analysis, the shape measure was recoded as a binary variable with “round-to-oval” as “0” and “other than round-to-oval” as “1”.

Relative centrality of the umbilical cord insertion was calculated from two variables recorded in the original data set. The distance from the cord insertion to the closest placental margin was recorded to the nearest cm. The type of umbilical cord insertion was coded as membranous (velamentous), marginal or normal (inserted onto the chorionic disk). We combined these two variables into a single distance measure, by recoding velamentous cord insertions as a negative value, cords inserted at the placental margin as “0” and progressively more central cords as “1” to “9” (overall scale range −13 to 13).

Estimated chorionic plate area was calculated as the area of an ellipse from two variables recorded in the original data set, the larger diameter and smaller diameter of the chorionic disc were recorded in cm. Disk eccentricity was calculated as the ratio of the larger and smaller diameters. Both the chorionic plate area and disk eccentricity could be cast as “interactions” between larger and smaller disk diameters.

Placental thickness at the center of the chorionic disc was recorded in units of 0.1 cm, by piercing the disc with a knitting needle on which millimeter marks were inscribed.

Placental weight was measured in decagrams to the nearest 10 grams; this variable was converted to Grams.

The fetoplacental weight ratio was calculated as birth weight divided by the placental weight, and is a value generally considered to reflect a physiologic state of balance between fetal and placental growth.

Umbilical cord length was analyzed as it was measured in the Labor and Delivery Room. Cord lengths ranged from seven to 98 cm.

Maternal characteristics were recorded at enrollment. Maternal age was coded as age at (enrollment) in years, and maternal height was measured in inches. Maternal weight prior to pregnancy was self-reported in pounds. Body mass index (BMI) was calculated from maternal height and weight. Parity counted all delivered live born offspring and did not include miscarriages/early pregnancy losses. Socioeconomic status index was a combined score for education, occupation and family income as scaled by the US Bureau of the Census. [21] Mother's race was coded as a binary variable denoting African-American as “1” and all others as “0”; original data coded race as Caucasian, African American, and “other”, most of whom were Puerto Ricans (9.2 percent). Cigarette use was coded by maternal self report at enrollment as non-smoker (coded as <1 cigarette per day), or by the self-reported number of cigarettes smoked daily grouped as 1-9, 10-20, and >20 (greater than one pack per day).

The allosteric metabolic equation was solved for estimates of α and [symbol] . Specifically, PW=α—(BW)̂ [symbol] is rewritten as a standard regression equation and solved for α and [symbol] :

Log(PW)=Log α+ [symbol] Log [(BW)] [Equation 1.1]

From Equation 1.1, Log α=Log(PW)− [symbol] [Log(BW)] [Equation 1.2);

Substituting the mean [symbol] for the population, this second equation was solved for each case, and the calculated Log α was exponentiated and used as a dependent variable in subsequent analyses, Spearman's rank correlations and multivariate regression were used to determine significant associations with P<0.05 was considered significant throughout. Three analyses were run. The first included all placental variables; thus the point-estimate of effect for each placental variable is adjusted for the presence of the others. The second included all maternal and fetal variables; again, data presented reflect effects adjusted for the presence of the other maternal variables. The third analysis included all variables (placental, maternal and fetal). Table 1 shows that the mean [symbol] was 0.78, ˜equal to the scaling of a fractal transport network.

TABLE 1
Descriptives of the placental measures (N = 24,061),

| | Overall Population | |
|---|---|---|
| | Mean (SD) | Range |
| a | −0.25 (0.17) | −1.23, 0.62 |
| | 0.78 (0.02} | 0.66, 0.89 |

Table 2 shows that each of the (crudely measured) placental dimensions altered the equation relating placental weight and birth weight.

TABLE 2
Placental, maternal and fetal influences on α

| Variable | | Multivariate model- | Multivariate model- | Multivariate |
|---|---|---|---|---|
| | | Placental variables | Maternal and fetal | model-All |
| Placental shape | Round-oval | −0.021 (0.005)*** | | −0.019 (0.005)*** |
| | Other than | | | |
| | round/oval | | | |
| Chorlonic olate area | | −0.001 (0.000)* | | −0.001 (0.000)*** |
| Disk elliosivity | | 0.167 (0.032)*** | | 0.158 (0.031)*** |
| Lar2er diameter | | 0.016 (0.004)*′′′* | | 0.030 (0.004)′′′** |
| Smaller diameter | | 0.042 (0.004)*** | | 0.054 (0.004)*** |
| Disc thickness | | 0.010 (0.000)*** | | 0.013 (0.000)*** |
| Cord lenKth | | 0.001 (0.000)*** | | 0.001 (0.000)*** |
| Relative cord | | 0.014 (0.007)* | | 0.008 (0.007) |
| Maternal a2e | | | 0.000 (0.000) | −0.001 (0.000)** |
| Parity | | | 0.000 (0.000) | 0.001 (0.001)* |
| Smokine: | | | 0.022 (0.001)*** | 0.017 (0.001)*** |
| Infant e:ender | | | 0.020 (0.002)*** | 0.018 (0.002)*** |
| Birth lene:th | | | 0.002 (0.000)*** | −0.011 (0.000)*** |
| Maternal BMI | | | [symbol] | [symbol] |
| Socioeconomic status | | | 0.000 (0.001) | [symbol] |
| African-American | | | 0.002 (0.003) | [symbol] |
| Gestational ae:e | | | [symbol] | [symbol] |

***P < 0.0001 bolded and italicized;
**P < 0.001;
*P < 0.05;
Not bolded, P > 0.05. .

In a modern data set with our more sensitive and valid methods of measuring placental shape, we were more direct. Using the population a derived from the Collaborative Perinatal Project, we solved for [symbol] and subtracted the calculated [symbol] from the population [symbol] , and explored the relationships between “delta [symbol] ” and the irregularity of the placental shape measured in 3 ways: 1. From the centroid of the placental shape (the mathematical center of the placenta, a physiologically arbitrary point); 2. From the site of umbilical cord insertion, the actual point of origin of the placental fractal vascular network; and 3. The roughness, calculated as the ratio of the perimeter to that of the smallest convex hull. Deviations from the ideal fractal scale were uncorrelated with the biologically arbitrary centroid, but were highly correlated with both the radial deviation from the umbilical cord insertion, and the roughness, a general measure of perimeter irregularity.

TABLE 3
Correlation of the deviation from a round shape with a
deviation from the 3/4 rule.

| | | Beta 3_4 |
|---|---|---|
| Radial standard deviation • | Pearson Correlation | .020 |
| of the plate area from the | | |
| centroid | | |
| | Significance | .485 |
| | N | 1199 |
| Radial standard deviation | Pearson Correlation | −.076 |
| of the plate areafrom the | | |
| umbilical cord | | |
| | Significance | .009 |
| | N | 1187 |
| Roughness = ratio of the | Pearson Correlation | .091 |
| perimeter to that of the | | |
| smallest convex hull | | |
| | Significance | .002 |
| | N | 1199 |

In another data set the blood vessels were traced on digital images of the placental chorionic surface. A distance measurement algorithm was applied to the image to determine the distance from each pixel to the nearest blood vessel. A metric was calculated using the mean distance divided by the placental diameter. Regression of that metric versus birth weight data showed that it accounted for 25% of birth weight variation.

### Example 5: Seven Slides

A set of 7 slides considered paradigms for major types of placental growth included: Normal placenta at 31 weeks (defined as prevalence of immature intermediate and mesenchymal villi, absent mature intermediate and terminal villi, poorly matured stem villi), Immature placenta at term (defined as prevalence of mature intermediate and stem villi, paucity of immature intermediate and terminal villi), Normal term placenta (defined as an even distribution of all types of villi), Preterm preeclampsia at 31 weeks (defined as poorly branched, extremely tiny, filiform terminal villi and an unusually wide intervillous space due to reduced terminal branching,), Term preeclampsia (defined as a generally even distribution of all villus types), and two cases of malformed villi with normal numerical mixture of villous types). A minimal set of villous morphometric algorithms developed with ECognition software was applied to these 7 slides.

Slide Digitization: Slides were digitized using an Aperio T3 instrument that is a self-contained system for image capture, manipulation and management. This included tissue finding, auto-focusing, automated scanning, image compression and slide quality assessment. All relevant image capture parameters (e.g., file name, ScanScope ID, scan time, barcode, quality score, the directory path to the virtual slide image, etc.) are stored in a Virtual Slide Manager database (Aperio, Vista, Calif.). The slides were stored as JPEG compatible .svs files for optimal computational speed within the ECognition framework.

The results showed that villous histologic features were reduced to 13 variables related to villous size and/or villous capillary location. At least 2 and as many as 5 variables significantly distinguished the abnormal patterns from the paradigm normal pattern (p<0.05).

TABLE 4
Factor means comparing pathology types to “normal”

| | Malformed | | | Preterm | Term |
|---|---|---|---|---|---|
| | at term | Preterm/immature | Immatureiterm | preeclampsia | preclampsia |
| Factor 1 | −0.08 v 0.15 | ..021 v. 0.13 | 0.04 v. .0.90 | 0.15 v. .076 | 0.13 v. 0.41 |
| Factor 2 | −0.26 v .0.45 | 0.04 v. −0.27 | 0.0 v .−0.14 | −0.14 v. 0.70 | 0.29 v. .0.96 |
| Factor 4 | −0.15 v .0.26 | −0.04 v. 0.21 | −0.02 v. 0.51 | .0.10 v. .0.50 | 0.05 v. −0.17 |
| Factor 6 | 0.09 v. −0.16 | 0.00 v. −0.01 | 0.01 v. −0.21 | .0.10 v. 0.48 | 0.00 v. 0.00 |
| Factor 7 | 0.04 v. −0.08 | 0.06 v .0.34 | 0.00 v. −0.04 | −0.03 v. 0.15 | −0.06 v. 0.19 |
| Factor 8 | 0.02 v. 0.04 | 0.02 v. .0.49 | | | |
| Factor 9 | −0.20 v. 0.04 | .0.20 v. 0.82 | | | |

In the 7 hematoxylin and eosin stained samples of placental villous branching morphogenesis types (paradigms for major types of placental growth), 80 variables were analyzed and reduced to 9 factors using principal components factor analysis (PCA) (see Table 4). The 6 paradigm patterns of abnormal placental villous branching were distinguishable from “term normal” by >1 factors, suggesting the present approach is tenable.

In the original test of 7 slides (see above), several variables could not be calculated; segmentation criteria were not robust to the full range of villous variability. Algorithms were revised and applied to 23 digitalized slides containing at least 1.5 MB of tissue data. 131 variables were calculated. Principal components analysis yielded 16 factors that together accounted for ˜88% of total data variance (see Table 5).

TABLE 5
PCA results showing Sfactors account for
2/30 f data variance

| | Initial Eigenvalues | | |
|---|---|---|---|
| Component | Total | % of | Cumulative |
| Factor 1 | 140.584 | 36.56 | 36.56 |
| Factor 2 | 11.518 | 10.38 | 6.94 |
| Factor 3 | 9.296 | 9.38 | 55.31 |
| Factor 4 | 7.386 | 6.65 | 61.97 |
| Factor 5 | .954 | 11.46 | 66.43 |
| Factor 6 | .293 | 3.87 | 70.30 |
| Factor 7 | 3.601 | 3.24 | 73.54 |
| Factor 8 | 3.190 | 2.87 | 76.42 |
| Factor 9 | 2.700 | 2.43 | 8.85 |
| Factor 10 | 1.841 | 1.66 | 80.51 |
| Factor 11 | 1.709 | 1.54 | 82.05 |
| Factor 12 | 1.551 | 1.40 | 83.45 |
| Factor 13 | 1.457 | 1.31 | 84.76 |
| Factor 14 | 1.251 | 1.13 | 85.89 |
| Factor 15 | 1.172 | 1.06 | 86.94 |
| Factor 16 | 1.114 | 1.00 | 87.95 |

Thus, automated assessment of placental villous branching growth is informative in clarifying placental pathology and by extension fetal pathophysiology.

### Example 6: Macroscopic Placental Measurement Tool

A random sample of 50 Kodachrome slides was obtained from Avon Longitudinal Study of Parents and Children (ALSPAC) and digitized using a computer linked Canon Canoscan FS2710. Images suitable for the graphical analysis methods were selected by a placental pathologist and epidemiologist.

A set of Excel-based macros were developed that capture and organize the mouse-clicks of a Kurta Graphics tablet. From digitized photographs of the placental chorionic surface, the umbilical cord insertion, the disk perimeter and terminal points of chorionic plate vasculature were marked (see e.g., FIG. 7). A second macro captured placental chorionic vasculature stereologically with a spiral grid of pitched at 1 cm intervals with the origin centered at the umbilical cord insertion. At each intersection of a placental chorionic vessel with the spiral, the sides of the vessel were marked, from which vessel numbers and calibers were calculated at distances from the umbilical cord insertion. A third macro traced the outlines of placental disk slices. The placenta was sliced in 8ths, creating 7 unique surfaces from which placental volume can be estimated without bias following Cavalieri's method. The macro also calculated mean and standard deviation of thickness, and minima and maxima relative to the cord insertion site and margins.

Standard regression analysis of placental chorionic surface characteristics was performed. The simple perimeter of the placental chorionic surface, oriented to cord insertion and disk edge closest to the site of membrane ruptured captured as much birth weight variance as placental weight. Novel measures accounted for more than twice the birth weight variance of current pathology standard measures (a single pair of diameters, and a single measure of disk thickness (c.f., Salafia et al, Am J Epidemiol 2005, November 15; 162(10):991-8)).

### Example 8: Testing Predictive Value for Abnormal Childhood Somatic Development

No comparable placental measures have been calculated previously in any of the national and international birth cohorts that have childhood follow-up. However, crude measures of the placental disk (a pair of placental chorionic disk diameters and one measure of disk thickness) were collected in the National Collaborative Perinatal Project (NCPP, recruited 1959-1966, see Salafia et al, Clin Obstet Gynecol. 2006 June; 49(2):236-56). Extracted was the first singleton liveborn of each family in the NCPP delivered at ≥34 gestational weeks (N=15,399). Body mass index (BMI) and IQ at age 7 years were regressed against z-scored placental weight, birth weight and estimated placental chorionic surface area (calculated from the larger and smaller placental disk diameters) and disk thickness. Placental chorionic surface area and disk thickness were independently associated with BMI and IQ at age 7 years after adjustment for birth and placental weights. These standard placental measures are not only crude but they more poorly measure more unusually shaped (and more poorly grown) placentas than more normal round, oval and uniformly thick placentas. Despite limitations, the above analysis demonstrates effects on both bodily growth and IQ at age 7 and supports the approach of using comprehensive placental measures to yield useful predictions of childhood health risks. Results are shown in the following Tables 6-9.

TABLE 6
Regression
Dependent Variable: Zscore Age 7 IQ
Predictors: Chronological age at the time of IQ test, Zscore chorionic
plate.area, Zscore placental thickness (.1 cms), Zscore cord length (cms),
Zscore birthweight, gms
Model Summary

| | | | Adjusted R | Std. Error of |
|---|---|---|---|---|
| 1 | .250 | .063 | .062 | .97170116 |

ANOVA

| Model | | Sum of | | | | |
|---|---|---|---|---|---|---|
| 1 | Regression | 1952.285 | 5 | 390.457 | 413.531 | .000(a) |
| | Residual | 29283.517 | 31014 | .944 | | |
| | | 3,235.802 | 31019 | | | |

Coefficients

| | | Unstandardized | | | |
|---|---|---|---|---|---|
| | | | Std. | | |
| 1 | (Constant) | .791 | .058 | 13.751 | .000 |
| | Zscore birthweight, gms | .076 | .006 | 11.853 | .000 |
| | Chronological age at time of test | −.002 | .000 | −12.977 | .000 |
| | Zscore placental thickness (.1 cms) | .118 | .006 | 20.466 | .000 |
| | Zscore chorionic plate area | .043 | .006 | 6.916 | .000 |

TABLE 7
Regression
Dependent Variable: Body Mass Index (BMI) at age 7 years
Predictors: (Constant), Zscore Placental thickness, Zscore Chorionic
plate area, Zscore length of cord (cms), Zscore birthweight (gms),
Zscore placental weight (gms)
Model Summary

| | | | Adjusted R | Std. Error of |
|---|---|---|---|---|
| Model | R | R Square | Square | the Estimate |
| 1 | .195 | .038 | .038 | 1.81252 |

ANOVA

| Model | | Sum of | | | | |
|---|---|---|---|---|---|---|
| 1 | Regression | 2000.113 | 5 | 400.023 | 121.8 | .000 |
| | Residual | 50569.281 | 15393 | 3.285 | | |
| | | 52569.394 | 15398 | | | |

Coefficients

| | | B | | | |
|---|---|---|---|---|---|
| 1 | (Constant) | 15.965 | .015 | 1093.013 | .000 |
| | Zscore placental weight | .055 | .022 | 2.511 | .012 |
| | (gms) | .074 | .015 | 4.898 | .000 |
| | Zscore length of cord (cms) | .242 | .019 | 12.946 | .000 |
| | | .059 | .019 | 3.157 | .002 |
| | | .068 | .016 | 4.184 | .000 |

TABLE 8
Dependent Variable: Zscore: Age 7 IQ.
Predictors: Chronological age at the time of IQ test, Zscore
gestational age at delivery in weeks, Zscore birthweight, gms,
log transformed score for fetal Inflammatory response in umbilical
cord, log transformed score for maternal inflammatory response
in extraplacental membranes and chorionic plate
Model Summary

| | | | Adjusted A | Std. Error of |
|---|---|---|---|---|
| 1 | .185(a) | .034 | .034 | .98736564 |

ANOVA

| Model f | | Sum of | | | | |
|---|---|---|---|---|---|---|
| 1 | Regression | 960.896 | 5 | 192.179 | 197.129 | .000 |
| | Residual | 27224.804 | 27926 | .975 | | |
| | | 28185.700 | 27931 | | | |

Coefficients

| | | Coefficients | | | |
|---|---|---|---|---|---|
| Model | | | Std. | t | Sia. |
| 1 | (Constant) | .782 | .061 | 12.765 | .000 |
| | Zscore birthweight, gms | .142 | .006 | 22.451 | .000 |
| | Chronological age at time | −.002 | .000 | −11.847 | .000 |
| | of test | | | | |
| | Zscore: gestational age at | .025 | .006 | 3.865 | .000 |
| | delivery, weeks | | | | |
| | Inflammatory response in | .240 | .020 | 12.182 | .000 |
| | umbilical | | | | |
| | log transformed score for | .152 | .014 | 10.629 | .000 |
| | maternal inflammatory | | | | |
| | response in chorionic | | | | |
| | plate | | | | |

TABLE 9
Dependent Variable: Zscore: Age 7 IQ; Predictors: Chronological
age at the time of IQ test, Zscore gestational age at delivery In
weeks, Zscore birthweight, gms, log transformed score for
infarct/abruption
Model Summary

| | | | Adjusted A | Std. Error of |
|---|---|---|---|---|
| 1 | .183 | .033 | .033 | .98683026 |

ANOVA

| Model | | Sum of | | | | |
|---|---|---|---|---|---|---|
| 1 | Regression | 1039.565 | 4 | 259.891 | 266.874 | .000(a) |
| | Residual | 30072.967 | 30881 | .974 | | |
| | | 31112.531 | 30885 | | | |

Coefficients

| | | Unstandardized | | | |
|---|---|---|---|---|---|
| Model | | B | Std. Error | | Sig. |
| 1 | (Constant) | .697 | .059 | 11.896 | .000 |
| | Zscore: birthweight gms | .145 | .006 | 24.235 | .000 |
| | Chronological age at | −.002 | .000 | −11.800 | .000 |
| | time test | | | | |
| | Zscore: gestation | .025 | .006 | 4.140 | .000 |
| | at delivery, | | | | |
| | log transformed score for | .108 | .008 | 13.578 | .000 |

### Example 9: Refinement

The data set is a 1,000 case subset of the Avon Longitudinal Study of Parents and Children (ALSPAC), an internationally recognized longitudinal study of children's health. The data set has been used to help understand the contribution of genetic factors, antenatal risk factors, peripartum conditions to perinatal and/or childhood outcomes. 14,000 placentas were collected and stored. The analytic sample used, and to be used, according to methods described herein include 1000 cases with placental photographs and a minimum of 7 tissue samples processed into wax blocks and H&E slides.

Macroscopic.

For macroscopic placental analysis, ALSPAC placental photographs, initially preserved as Kodachrome slides, are scanned using the Canon Canoscan FS2710 attached to a PC using Windows and stored as jpgs. Data are extracted from digitized images of placental chorionic surface and disk slices (see Example 3).

Additional data and orientation points are incorporated into the placental chorionic surface analyses (e.g., chorionic vascular branching and vessel calibers). As described above, the macros for chorionic vascular branching measurement have <5% inter-rater variability. Patterns of chorionic surface branching correlate with patterned villous branching in microscopic slides, and they combine to provide a measure that predicts the long term health of other organs that undergo contemporaneous (in utero) branching development. FIG. 8 shows an example of the tracing of chorionic vessel branching.

Disk thickness measures are incorporated into a single 3-dimensional macroscopic placental structural measurement model. Likewise, measurements of disk thickness (also with 5% inter-rater variability) that better capture its variability improve the ability to characterize placental structure. Moreover, differences in disk thickness may correlate with specific changes in microscopic patterned villous branching. Macroscopic and microscopic placental structural measures are likely to converge to identify abnormally stressful intrauterine environments. Data reductions by EFA/CFA and CN are compared, as for the microscopic tool.

Also, placental three-dimensional shapes are mathematically characterized, in terms of both chorionic plate area and disk thickness, as resulting from a single “disturbance” (or a “single hit”) and those with shapes that would require multiple “disturbances” (“multiple hits”), a recognized antecedent to poor outcome. Shapes are also analyzed in terms of the relative severity of “disturbances” required to generate achieved placental shapes. Because early (peri-implantation) placentas are thought to have a basic shape (discoid, centered about the umbilical cord insertion), abnormal shapes that result from maternal uteroplacental stressors or “disturbances” that deform normal uniform centripetal expansion are better able to be characterized.

Microscopic.

For microscopic placental analysis, H&E slides from ALSPAC are digitized using an Aperio T3 instrument.

### Example 10: Choronic Vascular “Fit”, Cord Centrality, and Fetal Growth Restriction

The experiments described herein demonstrate that poor “fit” of the chorionic vessels to the chorionic plate area and asymmetric growth of vessels from the cord insertion are correlated with reduced fetal growth.

314 consecutive consenting mothers delivering singleton live-born infants had placentas collected, and digitally photographed and weighed. The perimeter of the chorionic disk was traced; cord insertion and the sites at which each chorionic vessel dived beneath the chorionic plate were marked (see e.g., FIG. 7). More specifically, a print of the photograph was placed on a Kurta Graphics tablet, overlaid with a transparent 1 cm grid. X,Y coordinates were captured at each intersection of the perimeter with the grid, and additionally at any points of inflection. Then a second set of mouse clicks marked the end points of all chorionic plate surface vessels. The two shapes were interpolated. The area and perimeter demarcated by the diving sites was calculated. An algorithm calculated areas, and the centroid, the weighted center of the area. The chorionic plate and the chorionic vascular area were essentially treated as a pair of shapes that should “fit”. “Fit” is reflected in the distance between centroids. Dimensionless ratios of chorionic vascular area/perimeter and chorionic disk area/perimeter, distance between centroids of the inner and outer areas (inter-centroid distance), and distance from cord insertion to the disk area centroid (FIG. 11) were analyzed with regression, with P<0.05 significant. Observed/expected birthweight ratio (0/E BW) was calculated from national 50th percentile standards adjusting for gestational age, race, gender and parity. See Table 10.

Results showed that each measure was associated with O/E BW (parea=0.17, pperimeter=0.18, pinter-centroid=−0.18, pcord-centroid=−0.14) (see Table 11). Reduced area and perimeter ratios as well as greater inter-centroid distance (P<0.004) were related to reduced placental weight. In multiple regression, intercentroid and cord-centroid distances) retained independent effects on O/E BW (p=0.015, p=0.04). In a multivariate regression, the novel ratio measures accounted for 17% of O/E BW variance (r=0.44). Only intercentroid distance affected O/E BW independent of adjusted placental weight.

Area and perimeter ratios were normally distributed; inter-centroid and cord-centroid distances were skewed (see e.g., FIG. 12 C). Each was transformed and tested in univariate and multivariate regression. Only the intercentroid distance (“inintercent”) affected O/E BW ratio independent of placental weight (see Table 12).

Chattanooga babies tended to be slightly smaller than expected (mean=0.98) (see e.g., FIG. 12 A). Chattanooga placentas tended to be slightly larger than expected (mean=1.02) (see e.g., FIG. 12 B).

TABLE 10
Distributions of Novel Chorionic Disk Measures and Ratios.
This data set was a consecutively collected series of singleton
placentas of liveborn, non-anomalous infants born at Erlanger
Hospital, Chattanooga, TN, July-August 2005. The series was
collected to explore environmental contributions to a LBW
epidemic that has led to county-wide LBW rates of 14+%.
LBW rates were 17% in samples herein, with PTO rates of 11%.

| | Min | Max | Mean | SD |
|---|---|---|---|---|
| Chorionic plate area | 0.08 | 435.69 | 257.89 | 60.58 |
| Chorionic plate | 0.05 | 306.92 | 162.05 | 49.51 |
| perimeter | | | | |
| Chorionic plat | 0.53 | 0.99 | 0.93 | 0.06 |
| compactness | | | | |
| Chorionic plate | 24.34 | 119.55 | 41.74 | 9.46 |
| standard deviation | | | | |
| Distance between | 0.01 | 3.34 | 0.56 | 0.38 |
| chorionic vascular | | | | |
| and chorionic plate | | | | |
| centroids | | | | |
| Distance between | 0 | 0.2 | 0.04 | 0.03 |
| chorionic vascular | | | | |
| and chorionic plate | | | | |
| centroids, normalized | | | | |
| for scaling | | | | |
| Distance from cord | 0.04 | 17.82 | 3.61 | 2.29 |
| insertion point to | | | | |
| outer centroid | | | | |
| Distance between | 0.01 | 1.09 | 0.23 | 0.15 |
| cord insertion point | | | | |
| and outer centroid, | | | | |
| normalized for | | | | |
| scaling | | | | |
| Ratio of chorionic | 0.33 | 0.93 | 0.65 | 0.09 |
| vascular and | | | | |
| chorionic plate areas | | | | |
| Ratio of chorionic | 0.13 | 16.6 | 0.37 | 0.93 |
| vascular area to | | | | |
| chorionic vascular | | | | |
| perimeter (“ruffling”) | | | | |
| Ratio of chorionic | 0.88 | 3.23 | 1.65 | 0.29 |
| plate area to | | | | |
| chorionic plate | | | | |
| perimeter (“ruffling”) | | | | |
| Placental weight | 248.84 | 920.83 | 465.56 | 104.1 |
| adjusted for | | | | |
| gestational age | | | | |
| Birth weight | 1783.4 | 5896.4 | 3052.4 | 474.4 |
| adjusted for | | | | |
| gestational age | | | | |
| Observed/expected | 0.47 | 2.58 | 1.02 | 0.25 |
| placental weight ratio | | | | |
| Observed/expected | 0.55 | 1.61 | 0.98 | 0.15 |
| birth weight, ratio | | | | |

TABLE 11
Regression: Dependent-Observed/Expected Birth weight ratio

| | B | Std. Error | t | Sig. |
|---|---|---|---|---|
| (Constant) | 0.34 | 0.39 | 0.86 | 0.9 |
| Ratio of chorionic vascular area to chorionic | −0.47 | 0.34 | −0.87 | 0.369 |
| vascular perimeter (“ruffling”) | | | | |
| Ratio of chorionic plate area to chorionic plate | 0.69 | 0.8 | 0.86 | 0.39 |
| perimeter (“ruffling”) | | | | |
| Chorionic plate radius from centroid standard | 0.00 | 0.00 | 0.82 | 0.42 |
| deviation | | | | |
| Chorionic vascular radius from centroid standard | 0.00 | 0.00 | 0.94 | 0.35 |
| deviation | | | | |
| Distance between chorionic vascular and | 0.33 | 0.14 | 2.35 | 0.02 |
| chorionic plate centroids | | | | |
| Distance between Inner and outer centroids, | −5.67 | 2.23 | −2.54 | 0.01 |
| normalized for scaling | | | | |
| Distance from cord insertion point to outer | −0.07 | 0.03 | 2.43 | 0.02 |
| centroid | | | | |
| Distance between cord insertion point and outer | −1.08 | 0.45 | −2.37 | 0.02 |
| centroid. normalized tor scaling | | | | |
| Chorionic plate area compactness | 0.34 | 0.25 | 1.39 | 0.17 |
| Chorionic vascular area compactness | −0.01 | 0.16 | −0.05 | 0.96 |

TABLE 12
Regression: Dependent-O/E BW ratio, Ln transformed predictors

| Model | B | Std. Error | T | Sig. |
|---|---|---|---|---|
| (Constant) | 0.464 | 0.059 | 7.807 | 0.000 |
| Inintercent | −0.045 | 0.016 | −2.826 | 0.005 |
| Incordcent | −0.013 | 0.012 | −1.089 | 0.277 |
| O_e_pq | 0.341 | 0.033 | 10.181 | 0.000 |

Thus, variations in O/E BW for a given placental weight can be explained, at least in part, by subtle alterations in the relationships among placental parameters (e.g., cord insertion, chorionic vessel growth and chorionic disk expansion). Asymmetric growth of chorionic vasculature relative to the underlying chorionic disk results in a relatively inefficient placenta that produces a smaller-than-expected infant.

### Example 11: Placental Volume and Gestational Age Affects Birth-Weight

Gestational age plays a large role in accounting for the variability in birth-weight data. It can explain 50% of birth-weight variability. Calculations of placenta volume, in combination with birth-weight, will account for an additional 8% of variability in placenta volume. This is an indication that the three-dimensional shape of a placenta is an important factor in human health.

TABLE 13
R2 values for the three regression models

| | R2 |
|---|---|
| Volume and gestational age interacting vs. birth-weight | 0.58 |
| Gestational age vs. birth-weight | 0.50 |

### LITERATURE CITED

1. Benirschke K, K. P., Architecture of Normal Villous Trees, Chapter 7, in Pathology of the Human Placenta. 2002, Springer Verlag: New York, N.Y. p. 116-154.
2. Kaufmann, P., T. M. Mayhew, and D. S. Charnock-Jones, Aspects of human fetoplacental vasculogenesis and angiogenesis. II. Changes during normal pregnancy. Placenta, 2004. 25(2-3): p. 114-26.
3. Demir, R., et al., Classification of human placental stem villi: review of structural and functional aspects. Microsc Res Tech, 1997. 38(1-2): p. 29-41.
4. Kosanke, G., et al., Branching patterns of human placental villous trees: perspectives of topological analysis. Placenta, 1993. 14(5): p. 591-604.
5. Kaufmann, P., D. K. Sen, and G. Schweikhart, Classification of human placental villi. I. Histology. Cell Tissue Res, 1979. 200(3): p. 409-23.
6. Kingdom, J., et al., Development of the placental vil/ous tree and its consequences for fetal growth. Eur J Obstet Gynecol Reprod Biol, 2000. 92(1): p. 35-43.
7. Charnock-Jones, D. S., P. Kaufmann, and T. M. Mayhew, Aspects of human fetoplacental vasculogenesis and angiogenesis. I. Molecular regulation. Placenta, 2004. 25(2-3): p. 103-13.
8. Mayhew, T. M., D. S. Charnock-Jones, and P. Kaufmann, Aspects of human fetoplacental vasculogenesis and angiogenesis. Ill. Changes in complicated pregnancies. Placenta, 2004. 25(2-3): p. 127-39.
9. Demir, R., et al., Fetal vasculogenesis and angiogenesis in human placental villi. Acta Anat (Basel), 1989. 136(3): p. 190-203.
10. Benirschke K, K. P., Basic Structure of the Villous Trees (Chapter 6), in The Pathology of the Placenta. 2002, Springer-Verlag: New York. p. 50-115.
11. Benirschke K, K. P., Angioarchitecture of Villi, in The Pathology of the Placenta. 2002, Springer-Verlag: New York. p. 134-140.
12. Grether, J. K., et al., Reliability of placental histology using archived specimens. Paediatr Perinat Epidemiol, 1999. 13(4): p. 489-95.
13. Khong, T. Y., et al., Observer reliability in assessing placental maturity by histology. J Clin Pathol, 1995. 48(5): p. 420-3.
14. Khong, T. Y., Placental vascular development and neonatal outcome. Semin Neonatal, 2004. 9(4): p. 255-63.
15. Jaddoe, V. W. and J. C. Witteman, Hypotheses on the fetal origins of adult diseases: contributions of epidemiological studies. Eur J Epidemiol, 2006. 21(2): p. 91-102.
16. D E Boo, H. and J. E. Harding, The developmental origins of adult disease (Barker) hypothesis. Aust N Z J Obstet Gynaecol, 2006. 46(1): p. 4-14.
17. Barker, D. J., The developmental origins of insulin resistance. Horm Res, 2005. 64 Suppl 3: p. 2-7.
18. Levitt, N. S. and E. V. Lambert, The foetal origins of the metabolic syndrome—a South African perspective. Cardiovasc JS Afr, 2002. 13(4): p. 179-80.
19. Barker, D. J., The fetal origins of type 2 diabetes mellitus. Ann Intern Med, 1999. 130(4 Pt 1): p. 322-4.
20. Adair, L. and D. Dahly, Developmental determinants of blood pressure in adults. Annu Rev Nutr, 2005. 25: p. 407-34.
21. Levitt, N. S., et al., Adult BM/and fat distribution but not height amplify the effect of low birthweight on insulin resistance and increased blood pressure in 20-year-old South Africans. Diabetologia, 2005. 48(6): p. 1118-25.
22. Levitt, N. S., et al., An inverse relation between blood pressure and birth weight among 5 year old children from Soweto, South Africa. J Epidemiol Community Health, 1999. 53(5): p. 264-8.
23. Barker, D. J., C. Osmond, and C. M. Law, The intrauterine and early postnatal origins of cardiovascular disease and chronic bronchitis. J Epidemiol Community Health, 1989. 43(3): p. 237-40.
24. Barker, D. J. and C. N. Martyn, The maternal and fetal origins of cardiovascular disease. J Epidemiol Community Health, 1992. 46(1): p. 8-11.
25. Tanis, B. C., et al., Dutch women with a low birth weight have an increased risk of myocardial infarction later in life: a case control study. Reprod Health, 2005. 2(1): p. 1.
26. Rich-Edwards, J. W., et al., Longitudinal study of birth weight and adult body mass index in predicting risk of coronary heart disease and stroke in women. Bmj, 2005. 330(7500): p. 1115.
27. Lawlor, D. A., et al., Birth weight is inversely associated with incident coronary heart disease and stroke among individuals born in the 1950s: findings from the Aberdeen Children of the 1950s prospective cohort study. Circulation, 2005. 112(10): p. 1414-8.
28. Cooper, C., et al., Review: developmental origins of osteoporotic fracture. Osteoporos Int, 2005.
29. Gluckman, P. O., et al., Life-long echoes—a critical analysis of the developmental origins of adult disease model. Biol Neonate, 2005. 87(2): p. 127-39.
30. Jasienska, G., et al., High ponderal index at birth predicts high estradiol levels in adult women. Am J Hum Biol, 2006. 18(1): p. 133-40.
31. Lagiou, P., et al., Diet during pregnancy and levels of maternal pregnancy hormones in relation to the risk of breast cancer in the offspring. Eur J Cancer Prev, 2006. 15(1): p. 20-6.
32. Lagiou, P., et al., Maternal height, pregnancy estriol and birth weight in reference to breast cancer risk in Boston and Shanghai. Int J Cancer, 2005. 117(3): p. 494-8.
33. Baik, I., et al., Association of fetal hormone levels with stem cell potential: evidence for early life roots of human cancer. Cancer Res, 2005. 65(1): p. 358-63.
34. Lagiou, P., et al., Birthweight differences between USA and China and their relevance to breast cancer aetiology. Int J Epidemiol, 2003. 32(2): p. 193-8.
35. Nilsen, T. I., et al., Birth size and subsequent risk for prostate cancer: a prospective population-based study in Norway. Int J Cancer, 2005. 113(6): p. 1002-4.
36. Asbury, K., J. F. Dunn, and R. Plomin, Birthweight-discordance and differences in early parenting relate to monozygotic twin differences in behaviour problems and academic achievement at age 7. Dev Sci, 2006. 9(2): p. F22-F31.
37. Bellingham-Young, D. A. and E. N. Adamson-Macedo, Prematurity and adult minor illness. Neuro Endocrinol Lett, 2004. 25 Suppl 1: p. 117-25.
38. Nilsson, E., et al., Fetal growth restriction and schizophrenia: a Swedish twin study. Twin Res Hum Genet, 2005. 8(4): p. 402-8.
39. Gunnell, D., et al., The association of fetal and childhood growth with risk of schizophrenia. Cohort study of 720,000 Swedish men and women. Schizophr Res, 2005. 79(2-3): p. 315-22.
40. Willinger, U., et al., Neurodevelopmental schizophrenia: obstetric complications, birth weight, premorbid social withdrawal and learning disabilities. Neuropsychobiology, 2001. 43(3): p. 163-9.
41. Talbert, D. G., Uterine flow velocity waveform shape as an indicator of maternal and placenta/development failure mechanisms: a model-based synthesizing approach. Ultrasound Obstet Gynecol, 1995. 6(4): p. 261-71.
42. Naeye, R., Disorders of the Placenta and Decidua, in Disorders of the Placenta, Fetus and Neonata. 1992, Mosby Year Book: Philadelphia, Pa. p. 129-134.
43. Benirschke K, K. P., Placental Shape Aberrations, Chapter 13, in Pathology of the Human Placenta. 2002, Springer Verlag: New York, N.Y. p. 401-404.
44. Naeye, R., Disorders of the Placenta and Decidua, in Disorders of the Placenta, Fetus and Neonata. 1992, Mosby Year Book: Philadelphia, Pa. p. 129-130.
45. Benirschke K, K. P., Classification of Villous Ma/development, Chapter 15, in Pathology of the Human Placenta. 2002, Springer Verlag: New York, N.Y. p. 437-460.
46. Kaufmann, P., et al., Cross-sectional features and three-dimensional structure of human placental villi. Placenta, 1987. 8(3): p. 235-47.
47. Schweikhart, G., P. Kaufmann, and T. Beck, Morphology of placental villi after premature delivery and its clinical relevance. Arch Gynecol, 1986. 239(2): p. 101-14.
48. Larsen, L. G., H. V. Clausen, and L. Jonsson, Stereologic examination of placentas from mothers who smoke during pregnancy. Am J Obstet Gynecol, 2002. 186(3): p. 531-7.
49. Mayhew, T. M., Changes in fetal capillaries during preplacental hypoxia: growth, shape remodelling and villous capillarization in placentae from high-altitude pregnancies. Placenta, 2003. 24(2-3): p. 191-8.
50. Reshetnikova, O. S., G. J. Burton, and O. V. Teleshova, Placental histomorphometry and morphometric diffusing capacity of the villous membrane in pregnancies complicated by maternal iron-deficiency anemia. Am J Obstet Gynecol, 1995. 173(3 Pt 1): p. 724-7.
51. Vickers, M. H., et al., Fetal origins of hyperphagia, obesity, and hypertension and postnatal amplification by hypercaloric nutrition. Am J Physiol Endocrinol Metab, 2000. 279(1): p. E83-7.
52. Stocker, C. J., J. R. Arch, and M. A. Cawthorne, Fetal origins of insulin resistance and obesity. Proc Nutr Soc, 2005. 64(2): p. 143-51.
53. McMillen, I. C., C. L. Adam, and B. S. Muhlhausler, Early origins of obesity: programming the appetite regulatory system. J Physiol, 2005. 565(Pt 1): p. 9-17.
54. Armitage, J. A., P. O. Taylor, and L. Poston, Experimental models of developmental programming: consequences of exposure to an energy rich diet during development. J Physiol, 2005. 565(Pt 1): p. 3-8.
55. Longo, M., et al., Fetal origins of adult vascular dysfunction in mice tacking endothelial nitric oxide synthase. Am J Physiol Regul Integr Comp Physiol, 2005. 288(5): p. R1114-21.
56. Horton, T. H., Fetal origins of developmental plasticity: animal models of induced fife history variation. Am J Hum Biol, 2005. 17(1): p. 34-43.
57. Bertram, C. E. and M. A. Hanson, Prenatal programming of postnatal endocrine responses by glucocorticoids. Reproduction, 2002. 124(4): p. 459-67.
58. Green, L. R., Programming of endocrine mechanisms of cardiovascular control and growth. J Soc Gynecol Investig, 2001. 8(2): p. 57-68.
59. McMillan, I. C. and J. S. Robinson, Developmental origins of the metabolic syndrome: prediction, plasticity, and programming. Physiol Rev, 2005. 85(2): p. 571-633.
60. Wu, G., et al., Maternal nutrition and fetal development. J Nutr, 2004. 134(9): p. 2169-72.
61. Pham, T. D., et al., Uteroplacental insufficiency increases apoptosis and alters p53 gene methylation in the full-term IUGR rat kidney. Am J Physiol Regul Integr Comp Physiol, 2003. 285(5): p. R962-70.
62. Seckl, J. R., Glucocorticoids, feto-placental 11 beta-hydroxysteroid dehydrogenase type 2, and the early life origins of adult disease. Steroids, 1997. 62(1): p. 89-94.
63. Sibley, C. P., et al., Placental phenotypes of intrauterine growth. Pediatr Res, 2005. 58(5): p. 827-32.
64. Randhawa, R. and P. Cohen, The role of the insulin-like growth factor system in prenatal growth. Mol Genet Metab, 2005. 86(1-2): p. 84-90.
65. Wallace, J. M., et al., Nutritionally mediated placental growth restriction in the growing adolescent: consequences for the fetus. Biol Reprod, 2004. 71(4): p. 1055-62.
66. Baschat, A. A. and K. Hecher, Fetal growth restriction due to placental disease. Semin Perinatol, 2004. 28(1): p. 67-80.
67. Resnik, R., Intrauterine growth restriction. Obstet Gynecol, 2002. 99(3): p. 490-6.
68. Morley, R., Fetal origins of adult disease. Semin Fetal Neonatal Med., 2006. 11(2): p. 73-78.
69. Lockwood, C. J., The diagnosis of preterm labor and the prediction of preterm delivery. Clin Obstet Gynecol, 1995. 38(4): p. 675-87.
70. Metzger, R. J. and M. A. Krasnow, Genetic control of branching morphogenesis. Science, 1999. 284(5420): p. 1635-9.
71. Yevtodiyenko, A. and J. V. Schmidt, Dlk1 expression marks developing endothelium and sites of branching morphogenesis in the mouse embryo and placenta. Dev Dyn, 2006.
72. le Noble, F., et al., Control of arterial branching morphogenesis in embryogenesis: go with the flow. Cardiovasc Res, 2005. 65(3): p. 619-28.
73. Warburton, D., et al., Molecular mechanisms of early lung specification and branching morphogenesis. Pediatr Res, 2005. 57(5 Pt 2): p. 26R-37R.
74. Hu, M. C. and N. D. Rosenblum, Genetic regulation of branching morphogenesis: lessons learned from loss-of-function phenotypes. Pediatr Res, 2003. 54(4): p. 433-8.
75. Ingelfinger, J. R. and L. L. Woods, Perinatal programming, renal development, and adult renal function. Am J Hypertens, 2002. 15(2 Pt 2): p. 46S-49S.
76. Miettinen, P. J., Epidermal growth factor receptor in mice and men—any applications to clinical practice? Ann Med, 1997. 29(6): p. 531-4.
77. Miettinen, P. J., et al., Epithelial immaturity and multiorgan failure in mice lacking epidermal growth factor receptor. Nature, 1995. 376(6538): p. 337-41.
78. Grenander, U., General Pattern Theory. 1993, Oxford, UK: Clarendon Press.
79. Amit, Y., Grenander, U. Piccioni, M., Structural image restoration through deformable templates. Journal of the American Statistical Association, 1991. 86: p. 376-387.
80. Grizzi, F. F., B. Chiriva-Internati, M. Muzzio, P-C, Estimate of Neovascular Tree Complexity by Microscopy Analysis, in Current Issues on Multidisciplinary Microscopy Research and Education. 2005, FORMATEX. p. 140-149.
81. Giles, J., Benoit Mandelbrot: father of fractals. Nature, 2004. 432(7015): p. 266-7.
82. Meisel, L. V., Generalized Mandelbrot rule for fractal sections. Physical Review. A, 1992. 45(2): p. 654-656.
83. Keipes, M., F. Ries, and M. Dicato, Of the British coastline and the interest of fractals in medicine. Biomed Pharmacother, 1993. 47(9): p. 409-15.
84. Porter, R., et al., A fractal analysis of pyramidal neurons in mammalian motor cortex. Neurosci Lett, 1991. 130(1): p. 112-6.
85. Mayhew, T. M., et al., Stereological investigation of placental morphology in pregnancies complicated by pre-eclampsia with and without intrauterine growth restriction. Placenta, 2003. 24(2-3): p. 219-26.
86. Byrne, B. M., Factor analytic models: viewing the structure of an assessment instrument from three perspectives. J Pers Assess, 2005. 85(1): p. 17-32.
87. Coste, J., et al., Methodological issues in determining the dimensionality of composite health measures using principal component analysis: case illustration and suggestions for practice. Qual Life Res, 2005. 14(3): p. 641-54.
88. Bentler, P. M. and J. A. Stein, Structural equation models in medical research. Stat Methods Med Res, 1992. 1(2): p. 159-81.
89. Pembrey, M., The Avon Longitudinal Study of Parents and Children (ALSPAC): a resource for genetic epidemiology. Eur J Endocrinol, 2004. 151 Suppl 3: p. U125-9.
90. Patel, R. R., T. J. Peters, and D. J. Murphy, Prenatal risk factors for Caesarean section. Analyses of the ALSPAC cohort of 12,944 women in England. Int J Epidemiol, 2005. 34(2): p. 353-67.
91. Headley, J., et al., Medication use during pregnancy: data from the Avon Longitudinal Study of Parents and Children. Eur J Clin Pharmacol, 2004. 60(5): p. 355-61.
92. Fergusson, D. M., L. J. Horwood, and K. Northstone, Maternal use of cannabis and pregnancy outcome. Bjog, 2002. 109(1): p. 21-7.
93. Golding, J., Outcome of pregnancy in diabetic women. More investigation is needed into whether control of diabetes is really poorer in England than Norway. Bmj, 2001. 322(7286): p. 614-5.
94. Dorosty, A. R., et al., Factors associated with early adiposity rebound. ALSPAC Study Team. Pediatrics, 2000. 105(5): p. 1115-8.
95. Rogers, I., et al., Financial difficulties, smoking habits, composition of the diet and birthweight in a population of pregnant women in the South West of England. ALSPAC Study Team. Avon Longitudinal Study of Pregnancy and Childhood. Eur J Clin Nutr, 1998. 52(4): p. 251-60.
96. Farrow, A., K. M. Shea, and R. E. Little, Birthweight of term infants and maternal occupation in a prospective cohort of pregnant women. The ALSPAC Study Team. Occup Environ Med, 1998. 55(1): p. 18-23.
97. Maitra, A., et al., Mode of delivery is not associated with asthma or atopy in childhood. Clin Exp Allergy, 2004. 34(9): p. 1349-55.
98. Golding, J., Children of the nineties. A longitudinal study of pregnancy and childhood based on the population of Avon (ALSPAC). West Engl Med J, 1990. 105(3): p. 80-2.
99. Carey, W. M., SC., Infant Temperament Questionnaire (4-8 months). 1977, Philadelphia: Dept. Educational Psychology, Temple University.
100. Fullard, W. M., S C. Carey, W B., Toddler Temperament Scale (13 year old children). 1978, Philadelphia, Pa.: Dept. Educational Psychology, Temple University.
101. Buss, A. P., R., The EAS Temperament Scale., in Temperament: Early Developing Personality Traits. 1984, Erlbaum: Hillsdale, N.J.
102. Goodman, R., The Strengths and Difficulties Questionnaire: a research note. J Child Psychol Psychiatr 1997. 38: p. 581-586.
103. Frankenburg, W. D., J B., Denver Developmental Screening Test. J Pediatr 1967. 71: p. 181-191.
104. Griffiths, R., The abilities of babies. 954, New York: McGraw-Hill.
105. Fenson, L. D., P S. Reznic, S. et al., Technical Manual for the MacArthur Communicative Development Inventories. 1991, San Diego, Calif.: Developmental Psychology Laboratory.
106. Miller, M., Christensen, G., Amit, Y. Grenander, U., A mathematical textbook of deformable neuro-anatomies. Proceedings of the National Academy of Sciences, 1993. R90: p. 11944-19948.
107. Hastie, T., Tibshirani, R. Friedman, J H., Elements of Statistical Learning: Data Mining, Inference, and Prediction. 2001, New York: Springer.
108. Penev, P. A., J J., Local feature analysis: a general statistical theory for object representation. Network: Computation in Neural Systems, 1996. 7: p. 477-500.
109. Small, C., The Statistical Theory of Shape. 1996, New York: Springer.
110. Dryden, I. L. M., K V. , Statistical Shape Analysis. 1998, New York: Wiley Press.
111. Lele, S. R., J T., An Invariant Approach to Statistical Analysis of Shapes. 2000, London, UK.: Chapman and Hall/CRC Press.
112. McKeague, I., A Statistical Model for Signature Verification. Journal of the American Statistical Association, 2005. 100: p. 231-241.
113. Benirschke K, K. P., Normative Values and Tables (Chapter 28), in Pathology of the Human Placenta. 2002, Springer-Verlag: New York. p. 920-927.
114. Salafia, C. M., et al., Relationship between placental histologic features and umbilical cord blood gases in preterm gestations. Am J Obstet Gynecol, 1995. 173(4): p. 1058-64.
115. Salafia, C. M., et al., Intrauterine growth restriction in infants of less than thirty-two weeks' gestation: associated placental pathologic features. Am J Obstet Gynecol, 1995. 173(4): p. 1049-57.
116. Salafia, C. M., et al., Maternal, placental, and neonatal associations with early germinal matrixlintraventricular hemorrhage in infants born before 32 weeks' gestation. Am J Perinatal, 1995. 12(6): p. 429-36.
117. Salafia, C. M., et al., Clinical correlations of patterns of placental pathology in preterm pre-eclampsia. Placenta, 1998. 19(1): p. 67-72.
118. Salafia, C. M., et al., Placental pathologic features of preterm preeclampsia. Am J Obstet Gynecol, 1995. 173(4): p. 1097-105.
119. Salafia, C. M., et al., Placental pathology of absent and reversed end-diastolic flow in growth-restricted fetuses. Obstet Gynecol, 1997. 90(5): p. 830-6.
120. Viscardi, R. M. and C. C. Sun, Placental lesion multiplicity: risk factor for IUGR and neonatal cranial ultrasound abnormalities. Early Hum Dev, 2001. 62(1): p. 1-10.
121. Hagberg, H., D. Peebles, and C. Mallard, Models of white matter injury: comparison of infectious, hypoxic-ischemic, and excitotoxic insults. Ment Retard Dev Disabil Res Rev, 2002. 8(1): p. 30-8.
122. K, B., Examination of the Placenta, prepared for the Collaborative Study on Cerebral Palsy, Mental retardation and other Neurological and Sensory Disorders of Infancy and Childhood, N.l.o.N.D.a. Blindness, Editor. 1961, US Department of Health, Education and Welfare.
123. Kuh, D. and R. Hardy, A life course approach to women's health. Life course approach to adult health; no. 1. 2002, Oxford; New York: Oxford University Press. xvi, 419 p.
124. Niswander, K. and M. Gordon, The Collaborative Perinatal Study of the National Institute of Neurological Diseases and Stroke: The Women and Their Pregnancies. 1972, Philadelphia, Pa.: W. B. Saunders.
125. Myrianthopoulos, N. C. and K. S. French, An application of the U.S. Bureau of the Census socioeconomic index to a large, diversified patient population. Soc Sci Med, 1968. 2(3): p. 283-99.

1. A method of predicting the potential for manifestation of various medical conditions by analyzing the placenta comprising: determining the need for early monitoring, intervention or potential treatment for medical conditions likely to manifest as a child grows older; selecting and identifying a sample of the placenta to analyze by computer applied mathematical algorithms, determining the proper capturing device to obtain a three-dimensional image of the sample; preparing said placental sample to be analyzed; obtaining a three-dimensional digital image of the chorionic surface of the placental sample by use of the selected capturing device; receiving the digital image in a computing device; manually correcting for errors inherent in digital image acquisition; selecting corrected digital image data to input into the computer for analysis; initiating the computer to perform an analysis on the corrected digital image data using one or more algorithms to determine the vascular structure of the placenta; and interpreting said vascular structure to determine the potential for manifestation of various medical conditions.

2. The method of claim 1 wherein the digital image is obtained by configuring said selected capturing device to use polarized light or a polarized filter, and preparing said placental sample includes staining or treating said placental sample to increase transparency.

3. The method of claim 1 wherein the vascular structure is further analyzed by one or more dedicated mathematical algorithms including image segmentation, branching metrics, fourier analysis, or other graph or network metrics that are used to assess the timing of an event or stress including infection to the developing placenta or fetus.

4. The method of claim 1 further including the step of analyzing the results of the determined timing of an event or stress by combining the placental measures into a dedicated predictive model and using said model to assess future health risks to a patient.

5. A method of analyzing a human placenta according to claim 1 further comprising: identifying a sample of the placenta for analysis wherein said sample to be analyzed is selected from the group comprising the entire placenta, portions of the placenta, the placental shape, the umbilical cord, or the membranes.

6. A method of analyzing a human placenta according to claim 1 wherein the proper capturing device to obtain the highest quality three-dimensional image of said sample is selected from the group consisting of film, a digital camera, a 3D scanner, confocal microscopy, or 3D printing in conjunction with a microscope having a resolution capable of magnification of 20-40× power.

7. A method of analyzing a human placenta according to claim 1 further comprising: cropping the three-dimensional image to make the algorithm calculations more accurate; removing glare by identifying pixels with intensities above a pre-determined threshold and excluding said pixels from the calculations, analyzing identified structures to determine whether said structures are connected or disjointed and resetting the digital data collected by the image to reflect the proper structure as determined by the physician, applying a top hat filter and dilating the region by several pixels, and using Laplace's equation to fill in the regions.

8. A method of analyzing a human placenta according to claim 1 further comprising: analyzing the data to quantify the effect of abnormal placental growth on the fetus and predicting abnormal function of organs that are undergoing branching growth at the same time as the placenta.

9. A method of analyzing the placenta by utilizing computer applied mathematical algorithms specifically designed for this method and applied to data selected, corrected, and prepared for analysis by a physician comprising: determining the need for early monitoring, intervention or potential treatment for medical conditions likely to manifest as a child grows older; selecting and identifying a sample of the placenta to analyze; determining the proper capturing device to obtain a three-dimensional image of the sample; preparing said placental sample to be analyzed; staining said placental sample; obtaining a three-dimensional digital image of the chorionic surface of the placental sample by use of the selected capturing device; receiving the digital image in a computing device; pre-processing the digital image by performing an analysis on the digital image including reviewing the digital image data and manipulating the captured digital image data to correct for errors inherent in digital image acquisition; deciding which corrected digital image data to input into the computer for analysis; initiating the computer to perform an analysis on the corrected digital image data using one or more algorithms to determine the vascular structure of the placenta, and interpreting said vascular structure to determine the potential for manifestation of various medical conditions.

10. The method of claim 9 wherein barium is used to stain said placental sample in order to improve contrast between the vasculare structures and surrounding tissues in order that the vasculare structure color turns lighter thereby facilitating separation of the vascular structure from said surrounding tissues for analysis.
